(19) United States
(12) Patent Application Publication
(10) Pub. No.: US 2012/0245403 A1
(43) Pub. Date: Sep. 27, 2012

(54) Insole Electromagnetic Therapy Device

(75) Inventor: John Martinez, Woodsboro, MD (US)

(73) Assignee: BIOELECTRONICS CORP., Frederick, MD (US)

(21) Appl. No.: 13/428,920

(22) Filed: Mar. 23, 2012

Related U.S. Application Data
(60) Provisional application No. 61/603,834, filed on Feb. 27, 2012.
(60) Provisional application No. 61/604,449, filed on Feb. 28, 2012.

(30) Foreign Application Priority Data
Mar. 24, 2011 (CN) 201120086354.4

(51) Int. Cl. A61N 2/02

(52) U.S. Cl. 600/13

(57) ABSTRACT

A device includes a therapeutic electromagnetic circuit configured to emit an electromagnetic field upon activation and a resilient shoe insole coating surrounding the therapeutic electromagnetic circuit, in which the therapeutic electromagnetic device has a circuit board, having an electromagnetic field generator thereon, an antenna, coupled to the circuit board and arranged to radiate the electromagnetic field generated by the electromagnetic field generator, a power source, coupled to the electromagnetic field generator via an activator, and the activator, in which the activator, when turned on, is configured to activate the electromagnetic field generator.

### CLAIM OF PRIORITY

This application claims the benefit of priority from the prior Chinese Patent Application Utility Model No. 201120086354.4, filed Mar. 24, 2011, and U.S. Provisional Patent Application Ser. Nos. 61/603,834, filed on Feb. 27, 2012, and 61/604,449, filed on Feb. 28, 2012, the entire contents of which are hereby incorporated by reference.

### TECHNICAL FIELD

The following description relates to a portable electromagnetic therapy device that influences the metabolic characteristics of living systems. The techniques may be used to therapeutically promote healing of tissue and treat diseases.

### BACKGROUND

Therapeutic value may be achieved by applying an electromagnetic field to injured bodily tissue. Application of a high-frequency electromagnetic field at a sufficiently low field strength may result in a beneficial effect on healing of the tissue.

In some cases effectiveness of the therapeutic effect of the electromagnetic field may be improved by extending the duration of application of the field. The power requirements of the applied field may be reduced and the effectiveness of the treatment increased by extending the treatment duration.

### SUMMARY OF THE DISCLOSURE

The present application teaches systems and techniques for applying an electromagnetic field to bodily tissue.

In one aspect, a portable electromagnetic therapy device for applying a therapeutic electromagnetic field is disclosed, including an electromagnetic field generator, which is coupled to an antenna that is arranged to radiate the electromagnetic field. A power source is coupled to the generator to provide power for the device and an activator is used to initiate radiation of the electromagnetic field. The therapeutic device is self-contained and portable and is disposed over a surface of bodily tissue such that the radiated electromagnetic field impinges upon the bodily tissue.

In another aspect, a device includes a therapeutic electromagnetic circuit configured to emit an electromagnetic field upon activation, in which the therapeutic electromagnetic device includes a circuit board, having an electromagnetic field generator thereon, an antenna, coupled to the circuit board and arranged to radiate the electromagnetic field generated by the electromagnetic field generator, a power source, coupled to the electromagnetic field generator via an activator, and the activator, when turned on, initiating the electromagnetic field generator, and a resilient insole coating surrounding the therapeutic electromagnetic circuit.

In another aspect, a system includes a recharging station and a device having a therapeutic electromagnetic circuit configured to emit an electromagnetic field upon activation, in which the therapeutic electromagnetic device includes a circuit board, having an electromagnetic field generator thereon, an antenna, coupled to the circuit board and arranged to radiate the electromagnetic field generated by the electromagnetic field generator, a power source, coupled to the electromagnetic field generator via an activator, and a resilient insole coating surrounding the therapeutic electromagnetic circuit, in which the recharging station is operable to recharge the device.

In some implementations, the recharging station includes a battery, and a first recharging coil coupled to the battery, in which, during operation of the recharging system, the first recharging coil is operable to emit an electromagnetic field to recharge the device.

In some implementations, the device comprises a second recharging coil to receive the electromagnetic field emitted by the first recharging coil.

In some implementations, the power source is a battery of less than approximately 10 VDC.

In some implementations, the device is a component of a therapeutic delivery system. The therapeutic delivery system includes a member from the group of a patch, a bandage, a pad, a brace, a strap, tape, adhesive and a cast.

In another aspect, a technique for applying a therapeutic electromagnetic field is facilitated by incorporating a power source, antenna and electromagnetic field generator within a portable and disposable package and affixing the device to bodily tissue. The device generates an electromagnetic field that induces an alternating current in the bodily tissue. In another implementation, the average available radiated power is less than approximately 1 milliwatt and the peak available radiated power density is less than 100 microwatts per square centimeter measured substantially at the surface of the tissue.

Some implementations of the systems and techniques described herein may provide one or more of the following advantages. The device may be suitable for prolonged use. The self-contained unit can encourage patient compliance. In some implementations the device may be placed directly over bodily tissue to provide electromagnetic therapy to the tissue. The device may be part of a therapeutic agent delivery system such as a patch, bandage, pad, brace, cast, or other tissue injury support device.

In another aspect, a method is disclosed for inducing electrical current in a bodily tissue by: (1) positioning a device described herein adjacent a bodily tissue of an individual; and (2) operating the device for a duration, at a frequency, and at a peak available radiated power density effective to induce electrical current in the bodily tissue, wherein the device is positioned relative to the individual such that the device induces electrical current in the bodily tissue without making conductive contact with the bodily tissue. In some implementations, the induction of electrical current in the bodily tissue reduces or eliminates a pain sensation in the individual.

In another aspect, a method is disclosed for treating an individual by: (1) positioning a device described herein adjacent a bodily tissue of an individual; and (2) operating the device for a duration, at a frequency, and at a peak available radiated power density effective to elicit a therapeutic response in the individual, wherein the device is positioned relative to the individual such that the device induces electrical current in a bodily tissue of the individual without making conductive contact with the bodily tissue.

In another aspect, a method is disclosed for treating an individual by: (1) providing a device containing an electromagnetic field generator; (2) positioning the device adjacent a bodily tissue of an individual; and (3) operating the device for a duration, at a frequency, and at a peak available radiated power density effective to elicit a therapeutic response in the individual, wherein the device is positioned relative to the individual such that the device induces electrical current in the bodily tissue of the individual without making conductive contact with the bodily tissue, and wherein the device effects a penetration of the induced current into the bodily tissue such that the therapeutic response is elicited at a depth of at least 2 cm in the bodily tissue. In some implementations, the therapeutic response is elicited at a depth of at least 3, 4, 5, or 6 cm in the bodily tissue. In other implementations, the therapeutic response is elicited at a depth of 2 to 3, 2 to 4, 2 to 5, 2 to 6, 3 to 4, 3 to 5, or 3 to 6 cm in the bodily tissue.

In another aspect, a method is disclosed for treatment by: (1) providing a device selected from the group consisting of a pulsed electromagnetic field therapy (PEMF) apparatus, a transcutaneous electrical neural stimulator, and a static magnet array; (2) positioning the device at a distance from an individual effective to elicit a therapeutic response in the individual, wherein the device is positioned at a bodily location selected from the group consisting of the external end of the elbow transverse crease, the depression at the lower border of the malleolus lateralis, below the lateral extremity of the clavicle at the level of the first intercostals space, between the fourth lumbar vertebra and the fifth lumbar vertebra or 1 inch to the right or left thereof horizontally, a depression anterior or inferior to the head of the fibula, about 1.5 inches above the medial border of the patella, and between the radius and the palmaris longus; and (3) maintaining the device at the bodily location for a duration effective to elicit the therapeutic response.

In the methods described herein, positioning a device adjacent a bodily tissue of an individual refers to placing the device close to the skin of the individual (e.g., within 0.5, 1, 2, 3, 4, 5, or 6 inches of the skin) or in contact with the skin. The device can be encapsulated in a material and still be considered adjacent a bodily tissue, so long as it operates in the manner described herein. The methods do not entail penetration of the skin by the device and/or the application of electrodes to the skin (e.g., the device induces current in a bodily tissue in the absence of an application of electrodes to the skin). Tissues that can receive the electrical current according to the methods described herein include, for example, the skin as well as tissues that underlay the skin (e.g., joints or bones).

An exemplary device for use in the methods described herein comprises: an electromagnetic field generator; an antenna coupled to the generator and arranged to radiate the electromagnetic field; a power source (e.g., a battery) coupled to the generator; and an activator to initiate radiation of the electromagnetic field, wherein the device is self-contained and portable. The antenna can optionally contain antenna conductors on a printed circuit board. In some implementations, the device additionally contains: an annular ring to surround the battery; and a wire wound around the annular ring. In some implementations, the annular ring has a stepped cross-section and a wire wound on a top and outer side of the annular ring coupled to the antenna conductors. In some implementations, the annular ring contains a ferrite ring. In some implementations, the annular ring contains an insulating-magnetic ring.

The current induced in the bodily tissue of an individual can be, for example, parallel or perpendicular to the direction of antenna conductors.

In some implementations of the methods, devices, and systems described herein, the frequency is 27+/−0.5 MHz (e.g., 27.1 MHz).

In some implementations of the methods, devices, and systems described herein, the peak available radiated power density is less than 100 microwatts per square centimeter measured at the surface of the bodily tissue (e.g., the skin of the individual).

The device used in the methods can optionally contain a delivery system, e.g., a patch, bandage, pad, brace, strap, tape, adhesive, or cast. In some implementations the delivery system is a single use adhesive bandage.

The methods described herein can additionally include pulsing the generated electromagnetic field. In addition, the methods can also include altering at least one of a duty-cycle and a pulse repetition rate of the pulsed electromagnetic field. In some implementations, the duty cycle is approximately 8%-10%.

Certain=implementations include a portable electromagnetic therapy device, comprising: a circuit board, having an electromagnetic field generator thereon; an antenna coupled to the circuit board and arranged to radiate the electromagnetic field generated by the electromagnetic field generator; a power source, coupled to the electromagnetic field generator via an activator; and the activator, when turned on, initiating the electromagnetic field generator.

Preferably, the antenna is a sing loop wire, and may have either an asymmetrical shape or a symmetrical shape. For example, the single loop wire may have a shape selected from a group consisted of a circle, an ellipse, and a rectangle.

Various implementations may have various mechanical structures, preferably, the device is constructed by sequentially stacking hard potted enclosure layer, PCB layer, metal dome switches, and thin film layer substrate from the top down, wherein the PCB layer has the circuit board and the power source thereon, and the metal dome switches serve as the activator.

Preferably, the hard potted enclosure layer is made of epoxy or hard injection mold plastic. The PCB layer may also have an indicator indicating the status of the portable electromagnetic therapy device thereon. Preferably the part of the hard potted enclosure layer corresponding to the indicator is transparent.

Preferably, on the thin film layer substrate there are: an additional off switch cut for a separate metalized dome switch; a metalized dome cavity for ON switch or ON/OFF switch; and two pull tab transverse slits, which are arranged on both sides of the metalized dome cavity along the longitudinal axis and used for inserting a pull tab. The bottom of the PCB layer may contain shorting pads for ON switch and OFF switch, or just one shorting pad to toggle On/Off.

Moreover, the circuit board may be integrated into an ASIC chip to adapt to applications with compact size requirements. Preferably, on the thin film layer substrate there are: a metalized dome cavity for ON switch or ON/OFF switch; and two pull tab transverse slits, which are arranged on both sides of the metalized dome cavity along the longitudinal axis and used for inserting a pull tab. The bottom of the PCB layer may contain a shorting pad for ON switch, or just one shorting pad to toggle On/Off.

Preferably, the pull tab is inserted through the slits underneath the ON or ON/OFF switch metalized dome with its end extending out of the slits. Preferably, the pull tab is non-metallic.

Preferably, the sing loop wire has a length depending on the body site where the portable electromagnetic therapy device is applied and its characteristics including thickness, resistance, and material.

Preferably, the single loop wire has a diameter thickness of 0.8128 mm or 20 gauge, is circle-shaped, has a length ranging from 3.14 mm-47.12 cm, and is made of low resistance copper metal.

Preferably, the antenna is set on either side of the circuit board.

Preferably, the antenna is bendable to conform to the body curves of the body site where the portable electromagnetic therapy device is applied.

Preferably, the antenna is tightly encapsulated by an injection molded ring, and the injection molded ring is a semi-rigid ring.

Preferably, the circuit elements besides the antenna are sealed in a hardened moisture resistant enclosure. The thin film layer substrate may be made of a soft fabric and/or foam or other hygroscopic material.

Preferably, the activator is a key insert stick, configured for providing a temporary circuit shut off function by being inserted and circuit activation by being pulling out.

Preferably, the activator is one of a press switch assembly, a slide switch assembly, and a tactile press switch assembly. For example, the slide switch assembly may be constructed by stacking an injection molded switch channel, an injection molded switch cover, and a slide switch set on the circuit board. Preferably, there is a button clearance between the top surface of the injection molded switch channel and the top surface of the injection molded switch cover to protect the slide switch assembly from accidental activation. The button clearance is 0.05 mm to 25.4 mm.

Further, the slide switch assembly may also be constructed by stacking an injection molded thermoplastic elastomer outer shell, an injection molded button, an injection molded top cover, a slide switch set on the circuit board, and an injection molded bottom cover.

Besides, the tactile press switch assembly may be constructed by stacking a molded silicone rubber or injection molded thermoplastic elastomer outer shell, a momentary switch set on the circuit board, and a molded silicone rubber or injection molded thermoplastic elastomer bottom shell.

The portable electromagnetic therapy device may further comprise an indicator, indicating the status of the portable electromagnetic therapy device. For example, the indicator is a light-emitting diode, which transmits different lights depending on the status of the portable electromagnetic therapy device. Preferably the lights are visible and render different colors depending on the status of the portable electromagnetic therapy device. However, at least one of the lights may also be invisible and is picked up by a corresponding sensor.

The portable electromagnetic therapy device may further comprise a treatment timer. The light-emitting diode changes its luminosity as the timing of the treatment timer lapses.

Besides, the portable electromagnetic therapy device may include a battery decay circuit, which allows the light-emitting diode to change its luminosity as the battery decays.

Preferably, the portable electromagnetic therapy device may be placed into a soft bendable material to be wrapped around a body to maintain comfortable constant treatment. Further, the soft bendable material may be provided with a buckle to hold the back wrap ring module to the body. Preferably, straps and grommets are used to hold the device in place, and grommets are used to permanently hold the straps in place. Besides, the formed non-metallic ring is provided with prevention stubs and trough structure.

In some implementations, the individual has a pain-related disorder and the therapeutic response includes a reduction or elimination of pain in the individual. Examples of pain-related disorders include, for example, pain response elicited during tissue injury (e.g., inflammation, infection, and ischemia), pain associated with musculoskeletal disorders (e.g., joint pain such as that associated with arthritis, toothache, and headaches), pain associated with surgery, pain related to irritable bowel syndrome, and chest pain.

In some implementations, the individual has a disorder selected from the group consisting of adhesive capsulitis, tennis elbow, osteoarthritis, back pain, multiple sclerosis, tendon inflammation, and carpal tunnel syndrome, and the therapeutic response includes a reduction or elimination of pain associated with the disorder.

In some implementations, the individual has a bone, joint, soft-tissue, or connective tissue disorder and the therapeutic response includes a reduction or elimination of inflammation in a bone, joint, soft-tissue, or connective tissue of the individual. In some implementations, the individual has a bone, joint, soft-tissue, or connective tissue disorder and the therapeutic response includes a reduction or elimination of pain associated with the disorder.

In some implementations, the individual has a dental condition, and the therapeutic response includes a reduction or elimination of pain associated with the condition.

In some implementations, the individual has an arthritic disorder and the therapeutic response includes a reduction or elimination of pain associated with the disorder. In an example, the disorder is osteoarthritis of the knee and the therapeutic response includes a reduction or elimination of pain of the knee.

Details of one or more implementations are set forth in the accompanying drawings and the description below. Other features and advantages will be apparent from the description and drawings, and from the claims.

### BRIEF DESCRIPTION OF DRAWINGS

FIG. 1 is an implementation of a therapeutic electromagnetic device depicting an arrangement of the components.

FIG. 2 is an implementation of a therapeutic electromagnetic patch depicting components in layers.

FIG. 3 is a block diagram of an implementation of a therapeutic electromagnetic device.

FIGS. 4A-B illustrate a control waveform and resulting RF waveform.

FIGS. 5A-I illustrate alternative antenna configurations.

FIG. 6 depicts an alternative configuration of a therapeutic electromagnetic device.

FIGS. 7A-D depict various applications of a therapeutic electromagnetic device.

FIG. 8 is an implementation of an enhanced antenna.

FIG. 9 depicts anatomical locations for placement of a therapeutic device.

FIG. 10 depicts a simplified block diagram of a portable electromagnetic therapy device according to one implementation.

FIGS. 11A-C depict a main structure of a therapeutic electromagnetic device using antennas made of a single loop wire.

FIG. 12 depicts a sectional view of a slide switch assembly used as an activator.

FIG. 13 depicts an exploded view of the slide switch assembly shown in FIG. 12.

FIG. 14 depicts an exploded view of another slide switch assembly used as the activator.

FIG. 15 depicts an exploded view of a tactile press switch assembly used as the activator.

FIG. 16 depicts a main structure of a therapeutic electromagnetic device.

FIG. 17 depicts a detailed exploded view of a therapeutic electromagnetic device.

FIG. 18 depicts a detailed exploded view of a therapeutic electromagnetic device.

FIG. 19 depicts a detailed exploded view of a therapeutic electromagnetic device.

FIGS. 20A-B depict an encapsulating mechanism for a PCB, a top and bottom foam substrate, and a top pressure cap.

FIGS. 21A-E depict an assembling process of an On/OFF switch mechanism during manufacture.

FIGS. 21F-G depict an action mechanism for the On/Off switch during application.

FIG. 22 depicts an example of a therapeutic electromagnetic device.

FIGS. 23A-B respectively depict a side cross-section view and an exploded view of a therapeutic electromagnetic device, respectively.

FIGS. 24A-D respectively depict a top view, a bottom view, a top isometric view, and a bottom isometric view of a top plastic enclosure piece of a therapeutic electromagnetic device.

FIGS. 25A-D respectively depict a top view, a bottom view, a top isometric view, and a bottom isometric view of a bottom plastic enclosure piece of a therapeutic electromagnetic device.

FIG. 26 depicts a therapeutic electromagnetic device.

FIGS. 27A-B respectively depict a side cross-section view and an exploded view of a therapeutic electromagnetic device.

FIGS. 28A-C respectively depict a bottom view, a top isometric view, and a bottom isometric view of a top plastic enclosure piece of a therapeutic electromagnetic device.

FIGS. 29A-D respectively depict a top view, a bottom view, a top isometric view, and a bottom isometric view of a bottom plastic enclosure piece of a therapeutic electromagnetic device.

FIG. 30 depicts a top partial-cutaway view of a back wrap ring module, without a top housing, of a therapeutic electromagnetic device.

FIG. 31 depicts a bottom view of the back wrap ring module of a therapeutic electromagnetic device.

FIG. 32 depicts a top view of the fully assembled back wrap ring module of a therapeutic electromagnetic device.

FIG. 33 depicts internal details of the fully assembled back wrap ring module of a therapeutic electromagnetic device.

FIG. 34 depicts a back wrap ring module, placed into a soft bendable material of a therapeutic electromagnetic device.

FIG. 35 depicts a close-up view of the back wrap ring module placed in a soft bendable material of a therapeutic electromagnetic device.

FIG. 36 depicts an out waveform of an antenna of a therapeutic electromagnetic device.

FIG. 37A-37B are schematic representations of exemplary insole electromagnetic therapy devices.

FIG. 38 is a schematic representation of an exemplary preserver circuit.

FIG. 39 is a schematic circuit diagram of a preserver circuit and a receiver circuit.

FIG. 40 is a schematic representation of an exemplary pancake-style inductor coil.

FIG. 41 is a schematic representation of an exemplary printed circuit board on which a pancake foil is formed.

FIG. 42 is a schematic representation of an exemplary charger circuit.

### DETAILED DESCRIPTION

The systems and techniques described here relate to promoting therapeutic healing of tissue, providing prophylaxis for, and treatment of disorders and diseases through the application of an electromagnetic field. The techniques include providing a self-contained miniaturized electromagnetic field generating device that may be applied to bodily tissue. In some implementations the techniques and systems include devices that are disposable and portable.

The generated electromagnetic field can induce alternating current in bodily tissue. The alternating current may be subjected to non-linear electrical characteristics (for example, diode-like rectification) and so generate low frequency electrical potentials having a time dependence the same as the pulse modulation. The low frequency electrical potentials may stimulate cellular communication by, for example, altering the frequency of cellular activation potentials. Cellular communication may promote the healing of inflammation and the reduction of edema.

These techniques also may provide a method of transmission and utilization of the body's capacitance by affixing a transmitting element of the device to conform and fit closely over the bodily tissue, provide a small space and low weight device for field transport and emergency use. Patient compliance with a therapeutic regimen may be important to promote healing of bodily tissue. Patient compliance may be improved by providing a therapeutic device that is self-contained and portable.

Some or all of the components of a therapeutic electromagnetic energy delivery device may be integrated into a control circuit chip to miniaturize the device. The device may be affixed to various parts of the body for prolonged electromagnetic therapy. Patient compliance to the therapeutic regimen may be improved by embedding or concealing the device into a patch, bandage, pad, gel, wrap, brace, cast, or other injury support device. The device may be affixed to the body, taped over the bodily tissue, or placed in clothing worn by the patient.

The effectiveness of electromagnetic therapy may be improved by extending the treatment duration. Lower power electromagnetic radiation may be applied for a longer period of time than may be necessary for shorter periods of application. The self-contained unit disclosed might promote patient compliance with periods of therapy that may extend over weeks.

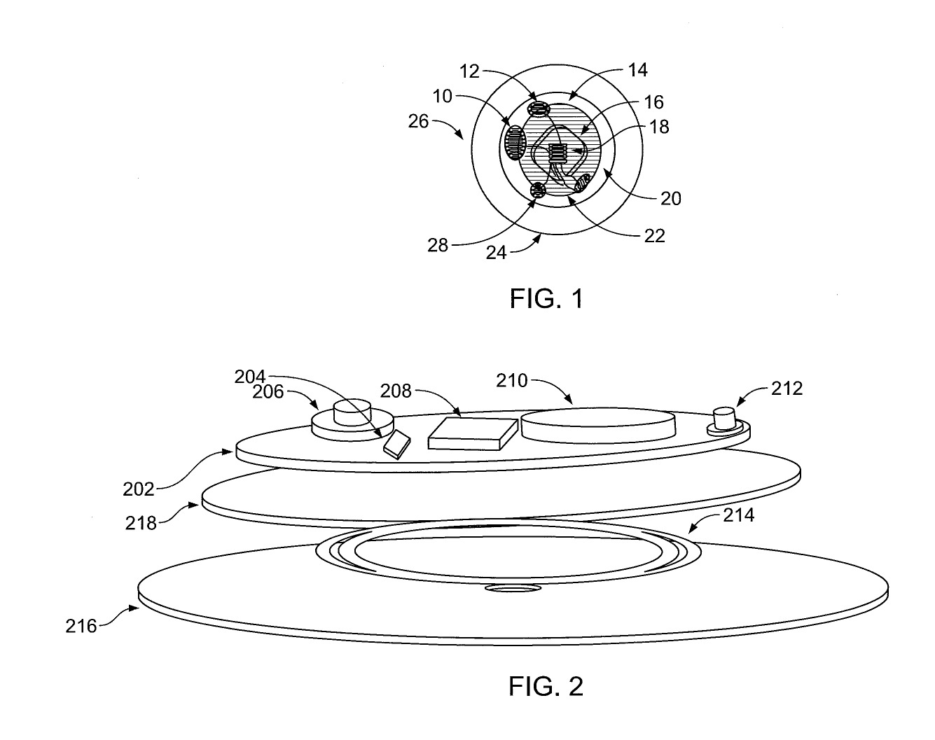

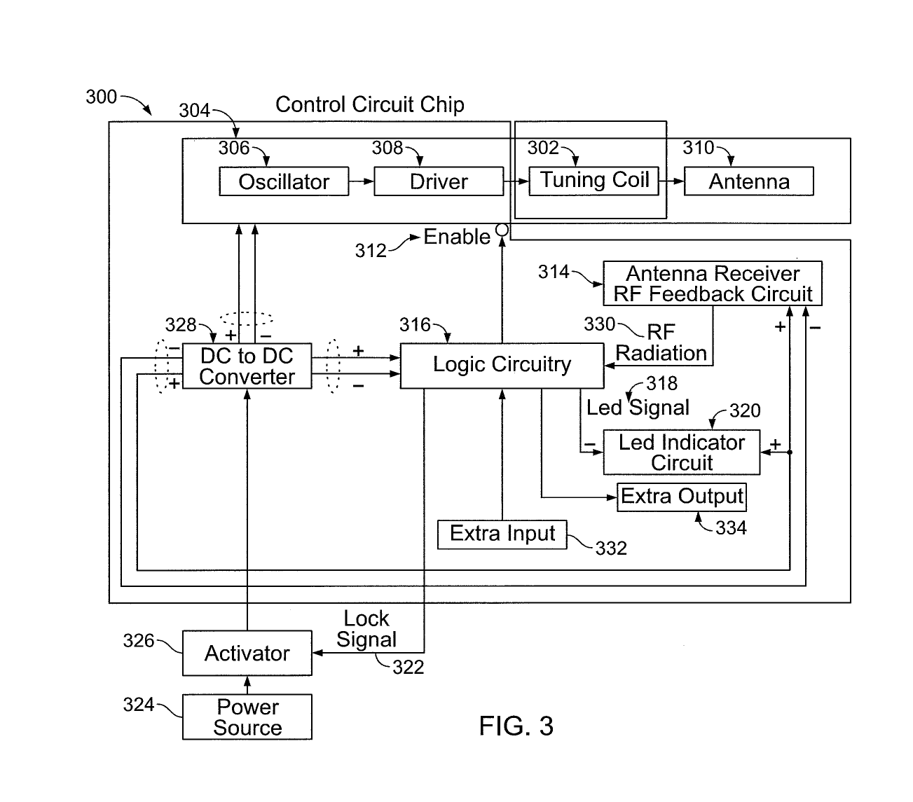

FIG. 1 illustrates an implementation of a therapeutic electromagnetic device 26. A control circuit chip 18 may provide the functionality for the therapeutic electromagnetic device to operate. An implementation of a control chip 18 is disclosed in association with the description of FIG. 3 and includes a radio frequency (RF) generator. A power source 10 coupled directly or indirectly to the control chip may be used to power the therapeutic electromagnetic device. The power source may include a battery, photovoltaic cell or an electro-chemical cell. An activator 12 is used to activate the device. The activator may include a switch that is a single-use or multiple use type and may be momentary or alternate-action. Actuation of the activator may be accomplished in various ways including by use of pressure, light or electronic signal either remotely or proximately. An antenna 16 is used to emit electromagnetic radiation and a deflector shield 14 may be used to deflect the electromagnetic radiation to the bodily tissue. In an implementation, the antenna 16 and/or deflector 14 may be tuned for electromagnetic energy in the frequency range of 27±0.5 Mhz. The therapeutic electromagnetic device also may include a tuning coil 20 which may be used to match the impedance of the antenna 16 to the RF signal generator within the control circuit chip 18. A circuit board 22 may be used to mount the elements of the device and, in some cases, provide coupling between the elements of the device. The circuit board may be comprised of a rigid or flexible material. The assembled device may weigh less than 12 grams.

In some implementations, a material 24 may be used for affixing the therapeutic electromagnetic device to bodily tissue. Material 24 can include, for example, pharmaceutical grade adhesives. The therapeutic electromagnetic device may be affixed using other single or multiple usage therapeutic delivery devices, which include a patch, a bandage, a pad, a brace, a strap, tape, adhesive and a cast.

In some implementations, an indicator 28 can be used to provide indicia that the therapeutic electromagnetic device is active. The indicator 28 may include one or more of the following: a visual indicator such as a light emitting diode (LED), lamp or electro-luminescent display; an auditory indicator such as noise generator; or a tactile indicator such as a vibrator. In an implementation, the indicator may be coupled to an electromagnetic field detector in the control circuit chip 18 and indicate the presence or lack of electromagnetic radiation from the device. In various implementations the indicator may be steady, intermittent or pulsed.

The therapeutic electromagnetic device may be enclosed or encapsulated in encapsulants or other potting compounds to reduce the vulnerability of the device to foreign materials including moisture, fluids, fungus, static charges, dirt, particulate matter and dust. The encapsulants, including insulating resins such as epoxies, polyurethanes, and polyesters, may be cast into cavities containing the device components, to insulate, protect, and hold the components in place. The encapsulant also may reduce the vulnerability of the device to environmental factors including air, heat, sunlight, ultraviolet light and spurious electromagnetic fields. In some implementations, a conformal coating may be applied to the device components and couplings to reduce the vulnerability of the device to moisture, fluids, fungus, static charges, dirt, particulate matter and dust. Alternatively, or in addition, the therapeutic electromagnetic device may be enclosed or encapsulated in encapsulants that provide resilience to large forces so the device may be used in locations where damage might otherwise occur. For example, the electromagnetic device may be enclosed in an encapsulant and used as an insole for shoes. The encapsulant can protect the device from damage that may otherwise occur when a patient uses the shoes for walking (e.g., by means of the force applied to the device from the patient's heel). For example, the encapsulant can include rubber or a gel, such as the gel used in Dr. Scholl's® gel insoles and inserts.

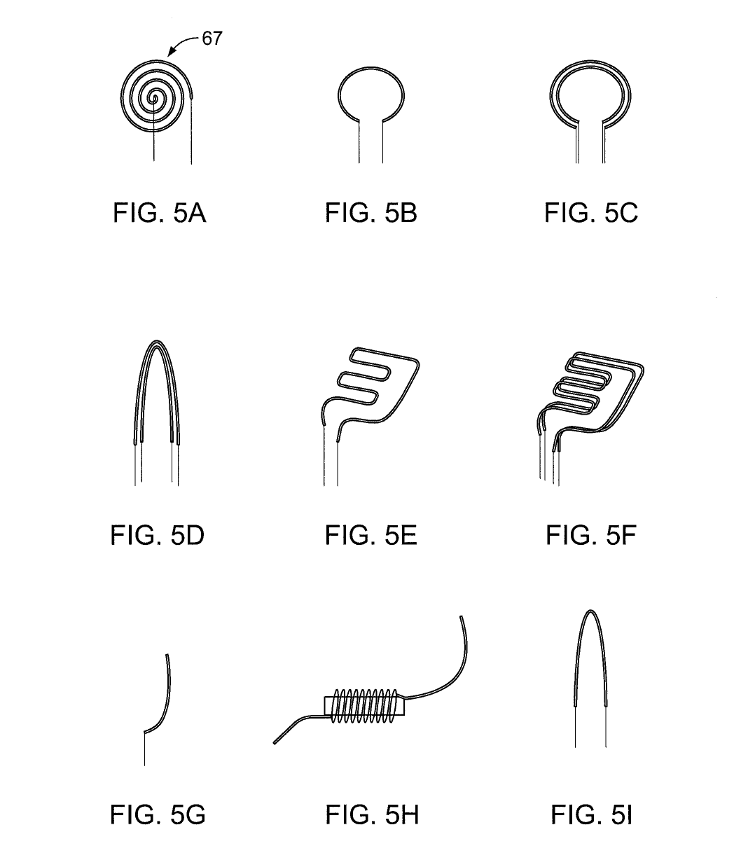

FIG. 2 illustrates an exploded view of an implementation of the therapeutic electromagnetic device having the components in a layered form. An activation switch 206, a control circuit chip 208, a power source 210, a visual indicator 212 and a tuning coil 204 may be mounted on a top layer and attached to a circuit board 202 to provide coupling between the components. A deflecting shield 218 may be layered under the circuit board 202. Or deflecting shield is a layer or coating of material, having high magnetic permeability, applied directly to circuit board 202. An antenna 214 to radiate electromagnetic energy may be layered under deflecting shield 218 and coupled to the circuit board 202. The deflecting shield 218 may deflect some of the energy radiated from the antenna 214 away from components mounted on the circuit board and toward the bodily tissue. The shape of the antenna is not restricted and some common shapes are depicted in FIGS. 5A-I. The antenna may also comprise separate conductors that do not make electrical contact with each other. In some implementations, the antenna may have a thickness of less than 5 millimeters and diameter of less than 9 centimeters or in other implementations, a length of less than 27 centimeters. The antenna may be incorporated into the circuit board 202.

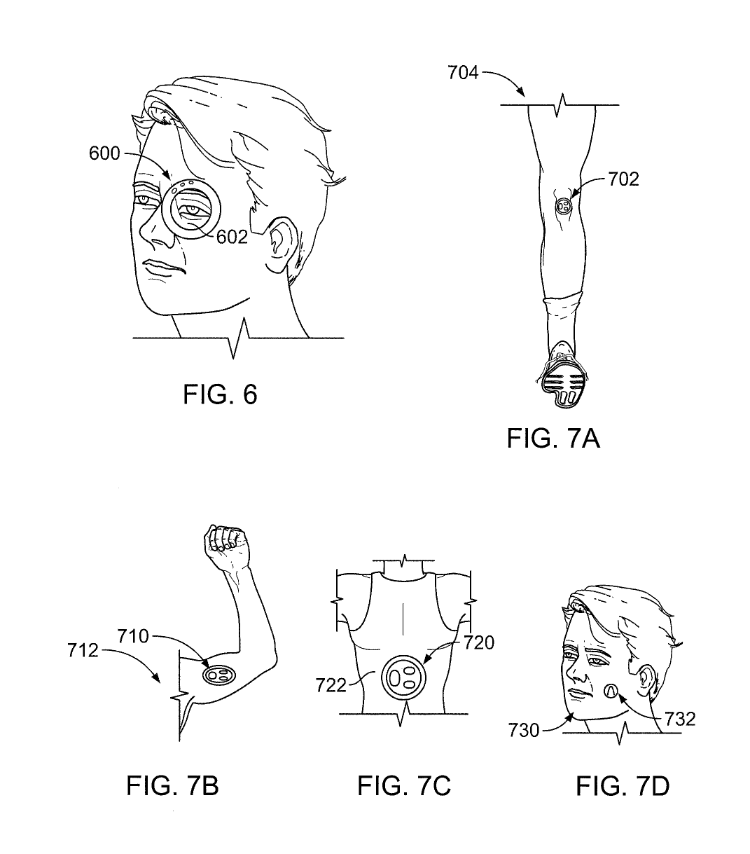

The shape of the circuit board 202 and deflecting shield 218 may be altered to adapt the therapeutic device to particular applications. The thickness of the device is less than 10 millimeters. In one implementation, an adhesive material 216 such as a pharmaceutical adhesive may be mounted to the bottom layer under antenna 214 to adhere the device to bodily tissue. Other therapeutic delivery devices including a patch, a bandage, a pad, a brace, a strap, tape, adhesive and a cast also may be used. In some implementations, the components may be selected and arranged for specific applications. Referring to FIG. 6, for example, the therapeutic device 600 may have a generally annular shape in a therapeutic application such as post-operative healing over an eye or breast. In this case, the annular shape defines a hole 602 through which a patient may see while the device is in place.

FIG. 3 is a block diagram of the circuitry of one implementation of a control circuit chip 300 used in a therapeutic electromagnetic device. Optionally, a tuning coil 302 may be included within the control circuit chip 300 or mounted separately. The components of the control circuit chip 300 may be integrated into one part or may be assembled from discrete components. The control circuit chip 300 includes an electromagnetic field generator 304 comprised of an oscillator 306 and a driver 308. Logic circuitry 316 coupled to the generator 304 provides an enable signal 312 to the generator 304. The logic circuitry also may provide an LED signal 318 to an indicator circuit 320, which, in turn, may be coupled to an indicator (not shown). Logic circuitry 316 may include discrete components, a programmable logic device (PLD), a microprocessor or other micro-controller unit (MCU). A power source 324 may be used to supply power to the electromagnetic therapy device. An activator 326 controls the flow of power from the power source to a DC-to-DC converter 328. The activator includes a switch that can provide for a one-time activation and then sustain activation for the duration of life of the power source. The DC-to-DC converter 328 provides power to the control chip components including the logic circuitry 316, the electromagnetic field generator 304 and an optional RF feedback circuit 314. The RF feedback circuit provides an RF radiation signal 330 to the logic circuitry 316. The logic circuitry also may provide an LED signal 318 to an LED indicator circuit and a lock signal 322 to the activator 326.

The electromagnetic field generator 304 comprises an oscillator 306 to generate an electromagnetic field, a driver circuit 308 to receive the electromagnetic field, amplify the wave and to provide the amplified wave to the optional tuning coil 302. The tuning coil 302 may be used to match the impedance of the driver 308 to an antenna 310, which is arranged to radiate the amplified electromagnetic energy. The oscillator 306 may be arranged to produce electromagnetic waves, including sinusoidal waves, at a carrier frequency of 27+/−0.5 megahertz (MHz). In an implementation, the electromagnetic therapeutic device has an average available power of less than approximately 1 milliwatt and a peak available radiated power density of less than 100 microwatts per square centimeter (μW/cm²) measured substantially at the surface of the tissue. The electrical efficiency of average available radiated power generation also may be greater than 20%. Average available power is the power that the device can dissipate into a resistive load. The average available power is distinguished from the power of the carrier within each pulse, which is termed the “peak” power. The peak available radiated power density is the maximum carrier wave power as if it was continuous and not pulsed, divided by the loop area of the antenna. A high voltage generator (not shown) may be included to increase the intensity of the radiated field. The high voltage generator may produce less than 30 VDC and may be synchronized to allow energy transforming action between therapy pulses, so that therapy pulses are not affected by the energy transformation action. Energy transformation could comprise connecting the battery to an inductive coil for a brief duration, and then switching the coil into a diode or rectifier and capacitor. The capacitor accumulates charge at a higher voltage than the battery. When voltage on the capacitor reaches a predetermined value, the capacitor may be discharged into the frequency generator for producing a therapy pulse. Alternatively, a transformer connected to a rectifier and capacitor as a flyback transformer may replace the inductive coil.

The enable signal 312 may be used to initiate or curtail radiation of the electromagnetic energy. The RF feedback circuit 314 is arranged to detect RF radiation from the antenna 310 and to provide RF radiation signal 330 to logic circuitry 316. Based on the level of the RF radiation signal 330, the logic circuitry provides the LED signal 318 to enable/disable the LED indicator circuit 320, which drives the indicator (not shown) and provides an indication that the antenna is radiating electromagnetic energy. The logic circuitry 316, the LED indicator circuit 320 or the indicator may be arranged so that the indicator is either indicating continuously, intermittently or pulsating. The logic circuitry also may provide the enable signal 312 to enable/disable the electromagnetic field generator 304.

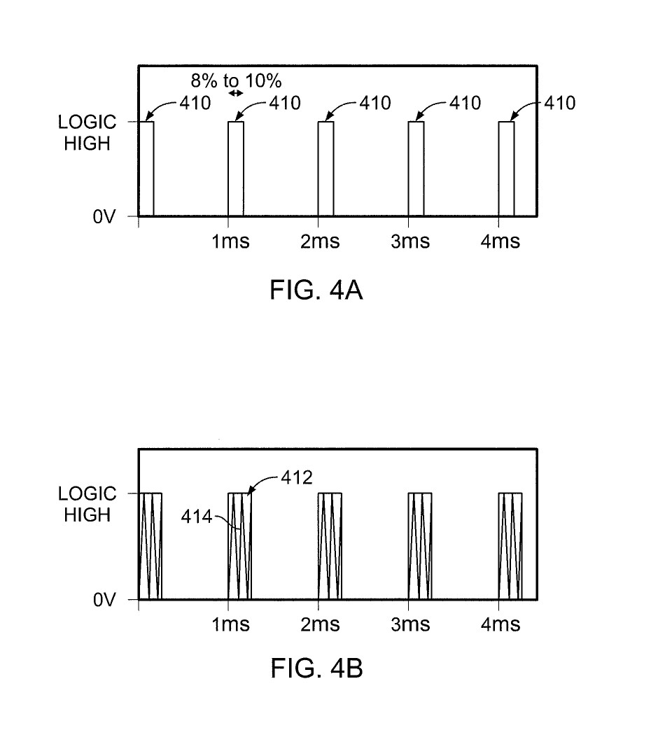

In an implementation, the energy radiated by the antenna 310 may be pulsed. PEMF may be used to provide electromagnetic field therapy over long periods of time and reduce heating of the bodily tissue. FIG. 4A illustrates that an enable signal 410 that may be provided from the logic circuit 316 to enable the generation and radiation of electromagnetic energy. In this example, the enable signal goes to a logic level high every millisecond. The enable pulse level is shown as logic high but alternatively may be logic low. In some implementations, the logic high level may be the power source, or regulated non-zero, voltage although other voltages are possible. The illustrated duty cycle is approximately 8% to 10%. In some implementations, the electromagnetic therapeutic device may operate in the frequency range of 3-30 MHz and application of the electromagnetic energy may be pulsed to maximize the therapeutic effect of the field. Pulses of 100 microsecond (μS) pulse duration at intervals of 1 millisecond (mS) (a pulse repetition rate of 1000 Hz) may be preferable. In order to reduce heating of the tissue, the electromagnetic field strength may be limited to less than 100 micro-Watts per square centimeter μWcm⁻²) as measured proximate the surface of the tissue. FIG. 4B illustrates a resulting output 412 from the antenna. The electromagnetic field 414 is radiated from the antenna only when the enable signal 410 is at logic high.

Referring again to FIG. 3, the power source 324 may be direct current (DC) and preferably less than approximately 10 VDC. The power source may be rechargeable. The rechargeable power source may be a battery of the lithium metal hydride or lithium ion or lithium polymer technology that may be recharged from an external source, including a sine wave field generator proximate the antenna 310 or separate coil (not shown) for the non-contacting induction of power from the external source into the therapeutic device. Current induced in the antenna or separate coil may be rectified and supplied as a reverse current to the rechargeable power source until the power source reaches a predetermined terminal voltage or case temperature.

The power source 324 is coupled to the activator 326. When the activator is actuated, power is coupled to the DC-to-DC converter, which may boost and regulate the power source voltage level. Regulated output voltage from the DC-to-DC converter 328 is supplied to the logic circuitry 316, electromagnetic field generator 304 and RF feedback circuit 314. A lock signal 322 may be provided by the logic circuitry 316 to lock the activator in the “on” position when the activator is actuated at least once.

Optionally, extra input signals 332 and extra output signals 334 may be received and/or provided by the logic circuitry 316 for additional functionality. For example, an output signal may be provided that provides indicia of the level of the voltage level of the power source 324. The output signal may activate a visual or auditory alarm when the power source requires replacement. An output signal may be provided that provides indicia of a state of the bodily tissue. The electrical permittivity and conductivity of tissue affects the frequency of the carrier wave in the device. The ratio of conductivity (σ) to permittivity multiplied by angular frequency (ωε) determines the polarity of the frequency change. If σ exceeds ωε then the carrier frequency decreases. If ωε exceeds σ then the carrier frequency increases. As conductivity is related to pH and free ion concentration, while permittivity is related to abundance of polar molecules and cell membrane charge, the bioelectrical state of the tissue may be assessed by determining the carrier frequency change from that at initial application of the device.

Optionally, the extra output signal 334 may provide control by enhancing the electromagnetic field for directed movement of chemical or pharmaceutical molecules in tissue, such as silver ions, for infection control. The enhanced electromagnetic field may be non-uniform in such a way as to direct movement of polar molecules, a method known as dielectrophoresis. Alternatively, the enhanced electromagnetic field may induce an electric field, which directs the movement of ions, a method known as iontophoresis.

An input 332 may be provided to receive external signals, for example, that alter the electromagnetic pulse duration, duty-cycle or pulse repetition rate of the electromagnetic field generated.

FIGS. 7A-D depict some applications of the therapeutic electromagnetic device. FIG. 7A depicts a therapeutic electromagnetic device affixed to a knee of a human leg 702. The device may be applied to aid in healing of, for example, a cracked knee, a cut, a sprain or strain. FIG. 7B depicts a therapeutic electromagnetic device 710 affixed to a muscle of a human arm 712 to aid in the healing of, for example, a sprain, a strain or a cut. FIG. 7C depicts a therapeutic electromagnetic device 720 affixed to a human abdomen 722 where, for example, lipo-suction procedures were performed. FIG. 7D depicts a human face 730 where a therapeutic electromagnetic device 732 is affixed on a left side of the face to aid in healing of an injury such as a tooth cavity.

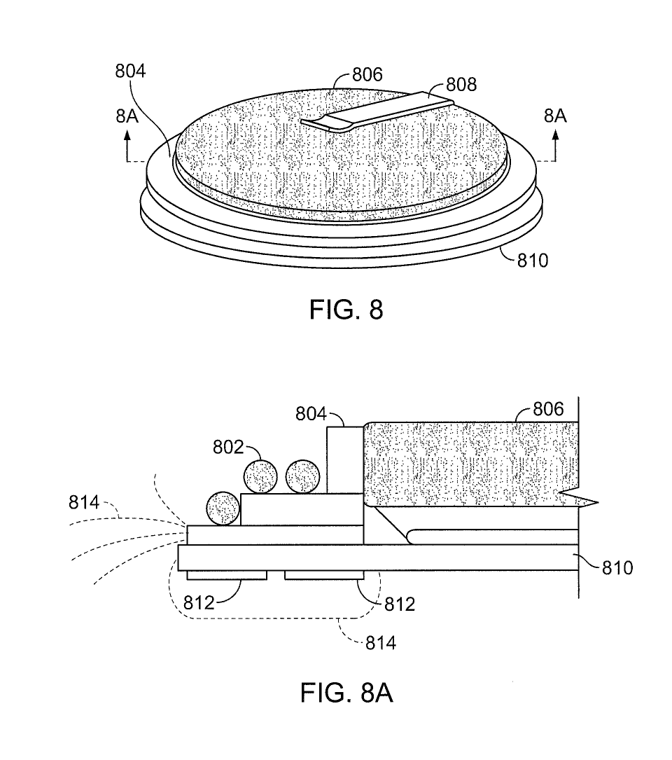

FIG. 8 depicts an implementation of an enhanced antenna comprising wires 802 wound around an annular ring 804 mounted on a printed circuit board 810. The ring may be a ferrite or magnetic, electrically-insulating ring. The ring may be arranged to support a battery 806 around the periphery. The battery 806 may be held in place by a retaining clip 808 to retain the battery adjacent the printed circuit board 810. Conductors 812 on the printed circuit board may be arranged to function as a main antenna for the therapeutic electromagnetic device and may be coupled to an electromagnetic field generator (not shown) as described above.

The annular turns of the wires 802 can convey current in phase and frequency with the main antenna 812. The number of turns of wire 802 on the annular ring are arranged to provide a larger magnetic flux than that of the main antenna 812. The windings cause a magnetic flux to enter/exit the outer perimeter of the annular ring. A portion of the (alternating) flux impinges bodily tissue underneath the therapeutic electromagnetic device inducing additional alternating current concentric with the main antenna. The additional induced current may result in magnetic flux that could otherwise be generated by a main antenna having a larger diameter. The magnetic field lines 814 from the main antenna conductors on the printed circuit board will take the path of least magnetic reluctance and pass around the underside of the printed circuit board. Only a weak magnetic field impinges the battery 806. The larger portion of the field may be restrained near the main antenna conductors. The effect is to generate increased magnetic field intensity farther in the bodily tissue. Thus, the main antenna, such as a simple loop antenna, with the enhanced antenna windings on the annular ring can present as an antenna with a larger effective diameter.

A simple loop antenna can produce a near field of electromagnetism, which can be confined within a certain volume by the physical geometry of the antenna. The magnetic field on the axis of a circular loop antenna diminishes in proportion to:

$$MagneticField \approx \frac{1}{{( 1 + {( \frac{z}{a} )}^{2} )}^{1.5}}$$

where z is the distance from the center of the loop and a is the radius of the loop. Beyond a distance Z, the current induced by the magnetic field in the bodily tissue may be ineffective to provide therapeutic value. The distance Z is measured at the point where the surface of the volume intersects the axis. A therapy volume wherein the electromagnetic field induced in the bodily tissue is adequate to have therapeutic value can be determined from the radius, and circularity, of the loop antenna and the current flowing in the antenna. Outside of this volume, therapy may be inadequate. Inside this volume, therapy may be effective and diminishing on approach to the surface of the therapy volume. In some implementations, the device effects a penetration of induced current into the bodily tissue such that a therapeutic response is elicited at a depth of at least 2 cm in the bodily tissue.

A larger effective diameter antenna can increase the magnitude of the induced current and extend the depth of penetration of induced current. Hence, the main antenna with the enhanced antenna may result in current induction inside the bodily tissue over a larger area and to a greater depth than with the main antenna alone.

### EXAMPLES

The therapeutic electromagnetic device mentioned above is generally portable, and may be applied to the body site needing treatment with various means, such as a patch, a bandage, a pad, a brace, a strap, a tape, an adhesive, an insole and a cast.

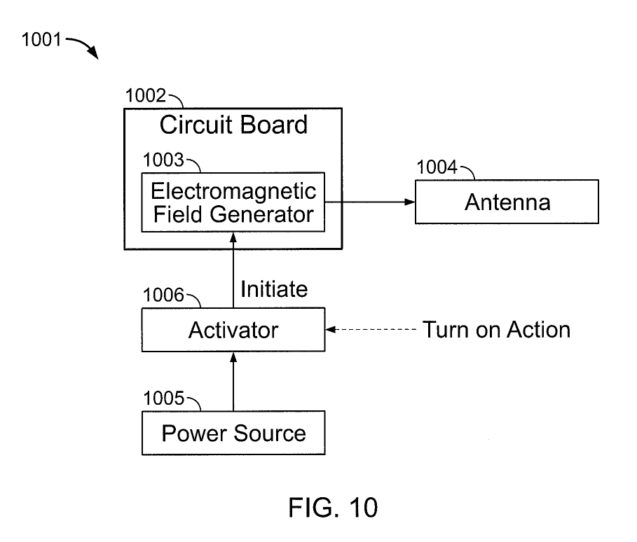

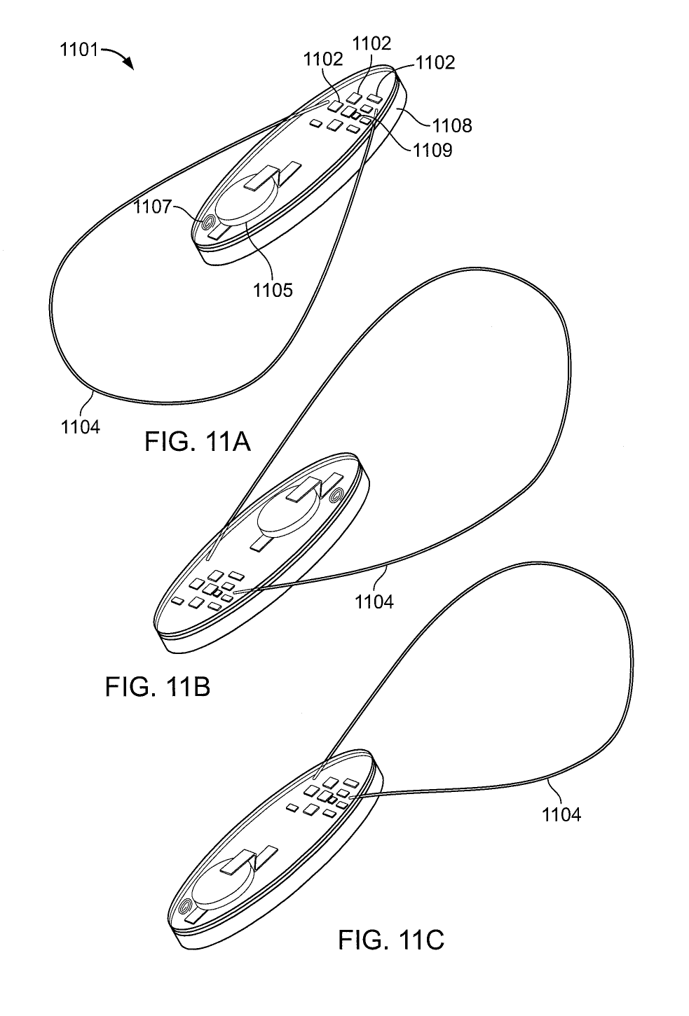

As shown in FIG. 10, an exemplary portable electromagnetic therapy device 1001 includes a circuit board 1002, having an electromagnetic field generator 1003 thereon; an antenna 1004, coupled to the circuit board and arranged to radiate the electromagnetic field generated by the electromagnetic field generator; a power source 1005, coupled to the electromagnetic field generator via an activator 1006; and the activator 1006, which, when turned on, initiates operation of the electromagnetic field generator.

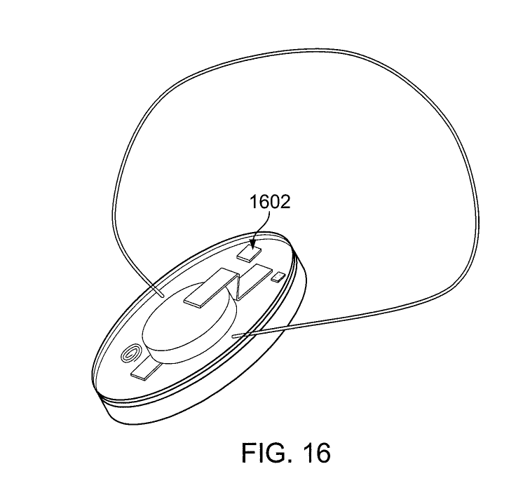

For example, the circuit board may be implemented with a control circuit chip as shown in FIG. 3, on a PCB 1102 as shown in FIG. 11A (referred to herein as “Model 071”), or on a ASIC chip 1602 for applications requiring compact-sized device as shown in FIG. 16 (referred to herein as “Model 088, femto product line”), depending on the particular applications of the portable electromagnetic therapy device.

Based on the electrical principle frame as shown in FIG. 10, different implementations may have different mechanical structures.

### First Examples

### Referred to Herein as “Models 071, 077/078,150”

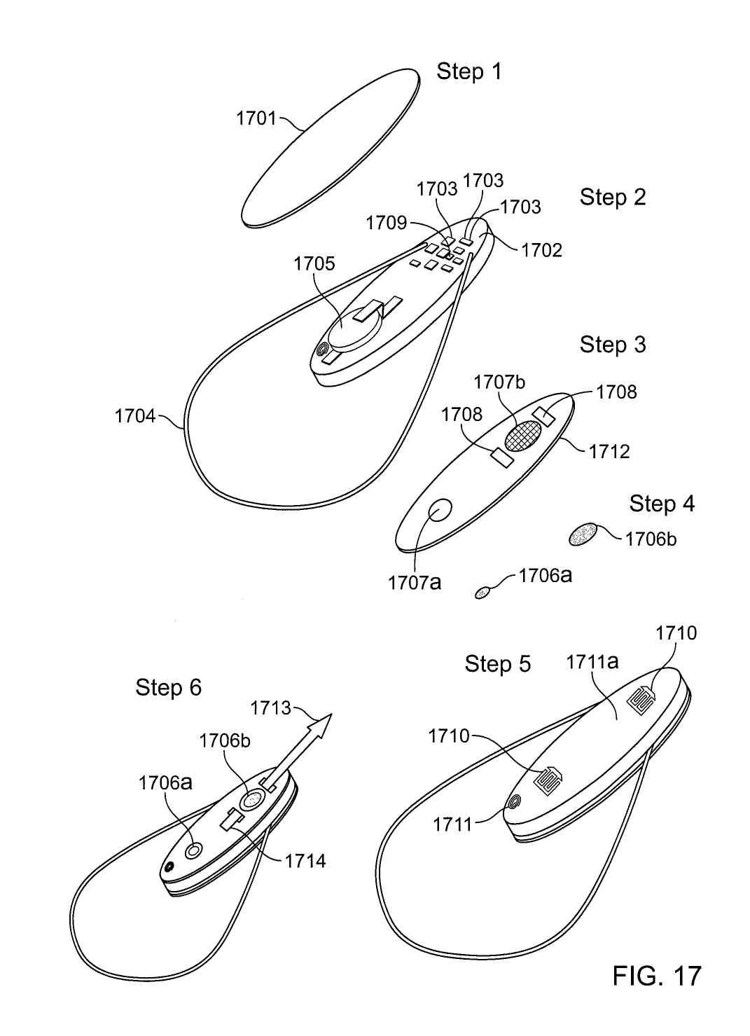

As shown in FIG. 17, implementations of the electromagnetic therapy device (Models 071, 077/078, and 150) can be constructed by sequentially stacking a hard potted enclosure layer 1701, a PCB layer including circuit elements 1703 (see the top of the PCB layer 1702 and bottom of the PCB layer 1711a), metal dome switches (off switch metal dome switch 1706a, and ON or ON/OFF switch metal dome switch 1706b), and a thin film layer substrate 1712 from the top down.

The hard potted enclosure layer 1701 may be made of materials such as epoxy or hard injection mold plastic, and protects the electrical elements on the PCB layer from the external environment. The top of the PCB layer 1702 has a LED visual indicator 1709, circuit elements 1703 including the electromagnetic field generator, and a battery 1705 with welded tabs thereon, coupled to a single wire antenna 1704. In a typical implementation, the part of the hard potted enclosure layer 1701 corresponding to the LED visual indicator 1709 is transparent so that the visual lights from the LED visual indicator may permeate such part and be seen by the user.

On the thin film layer substrate 1712 there are: an additional off switch cut 1707a for a separate metalized dome switch; a metalized dome cavity 1707b for ON switch or ON/OFF switch; and two pull tab transverse slits 1708, which are arranged on both sides of the metalized dome cavity 1707b along the longitudinal axis and can be used for inserting the pull tab 1713. Preferably, the pull tab 1713 is inserted through the slits 1708 underneath the ON switch metalized dome with its end 1714 extending out of the slits, in order to prevent accidental activation. In a typical implementation, the pull tab 1713 is not electrically conductive. In some implementations, the pull tab 1713 is non-metallic. The bottom of the PCB layer 1711a may contain shorting pads 1710 for ON switch and OFF switch, or just one shorting pad 1710 to toggle On/Off. The bottom of the PCB layer 1711a may additionally have preserver coil 1711 to implement On/Off function through induction.

Typically, the antenna 1104 in Model 077/078 (see FIG. 11B) is larger than that in Model 071 (see FIG. 11A).

### Second Example

### Referred to Herein as “Model 088”

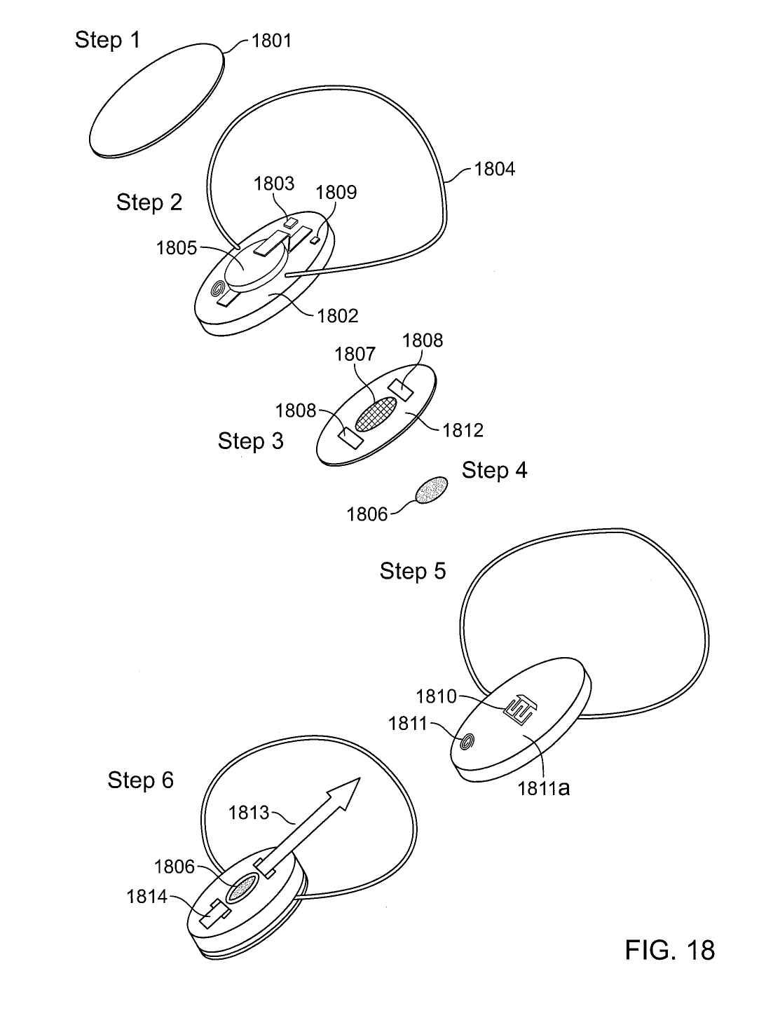

The example mechanical structure (Model 088) of the electromagnetic therapy device as shown in FIG. 18 is similar to the device shown in FIG. 17 (wherein similar reference numerals in FIGS. 17-18 refer to similar components), except that the circuit board in FIG. 18 is integrated into an ASIC chip 1803 and the additional off switch cut 1707a is absent. Preferably, the pull tab 1813 is inserted through the slits underneath the ON switch 1806 metalized dome with its end 1814 extending out of the slits, in order to prevent accidental activation. In some implementations, the pull tab 1813 is non-metallic. The bottom of the circuit board layer 1811a may contain a shorting pad 1810 for ON switch or just one shorting pad 1810 to toggle On/Off. The bottom of the PCB layer 1811a may additionally have preserver coil 1811 to turn the device ON or OFF through induction.

Compared to the example shown in FIG. 17, the example shown in FIG. 18 typically has a more compact size and is adapted to smaller physical sites.

### Third Example

### Referred to Herein as “Model 240”

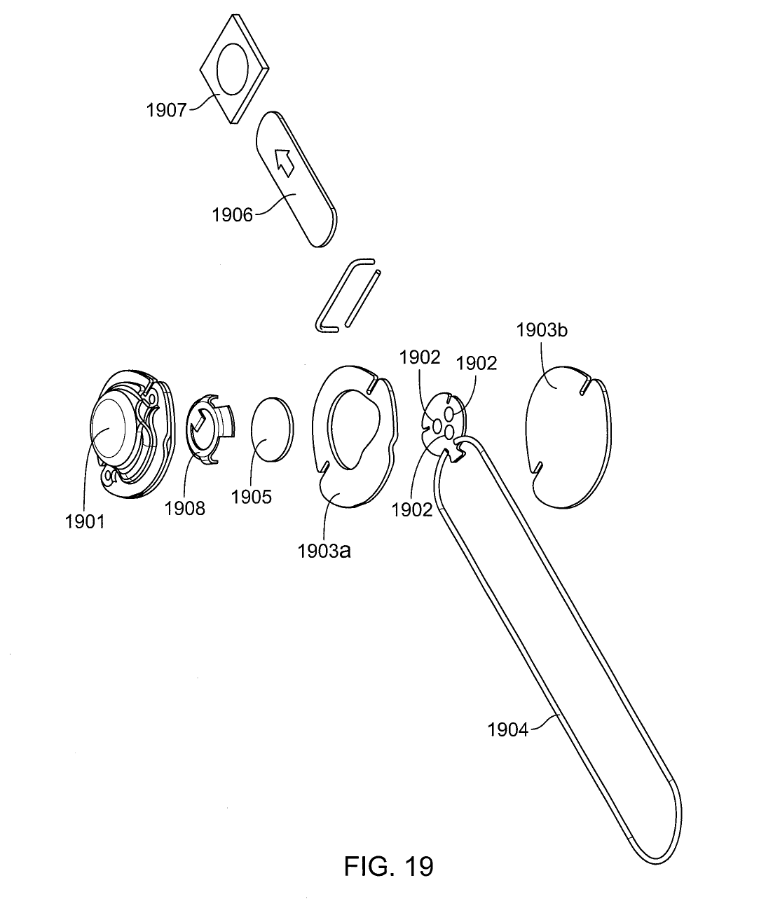

FIG. 19 depicts an exploded view of another example of the electromagnetic therapy device (Model 240). As shown in FIG. 19, the electromagnetic therapy device includes a PCB and electronic parts 1902, a single wire antenna 1904, a top pressure cap 1901, a battery 1905, and top and bottom foam substrates 1903a and 1903b.

The PCB and electronic parts 1902 have an electromagnetic field generator thereon and the single wire antenna 1904 is used to radiate the electromagnetic wave generated by the electromagnetic field generator. The battery 1905 is used to supply power to the PCB 1902. Further, the top and bottom foam substrates 1903a and 1903b are arranged at the top and bottom end of the PCB and electronic parts 1902, respectively, to separate it from contacting the external environment, prevent short circuit, and facilitate the disposing of the top pressure cap 1901.

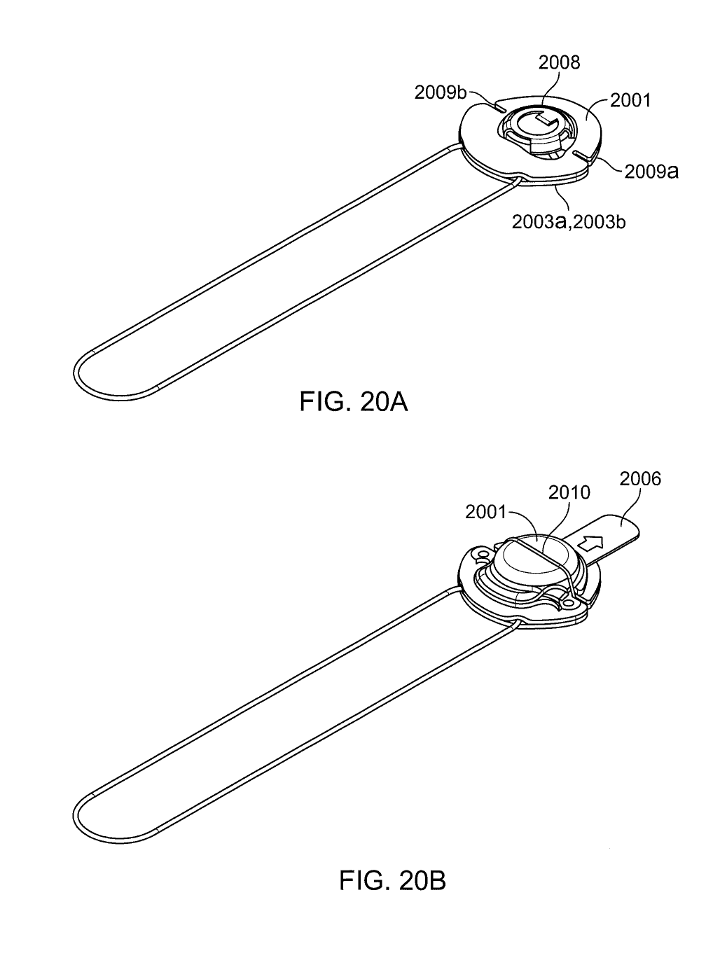

As shown in FIGS. 20A-B, both a top pressure cap 2001 and top and bottom foam substrates 2003a and 2003b have alignment cuts 2009a and 2009b respectively for a rubber band 2010, wherein in FIG. 20A the top pressure cap 2001 is removed for distinctly showing the alignment cut for the rubber band 2010. Upon aligning the corresponding alignment cuts 2009a and 2009b, rubber band 2010 may tightly coil through the alignment cuts 2009a and 2009b to encapsulate the top pressure cap 2001, PCB and electronic parts 1902, and the top and bottom foam substrates 2003a and 2003b together and thus hold the top pressure cap 2001 in place.

As shown in FIG. 19, the non metallic pull tab 1906 is inserted between the PCB 1902 and the battery 1905 as ex-factory product to completely packet the battery 1905 in the finished product, and is no longer used after pulling out. As a contrast, a non metallic key insert stick 1907 may be frequently used during the application of the product, and provides a temporary circuit shut off function when in place, but when it is removed, the circuit stays activated.

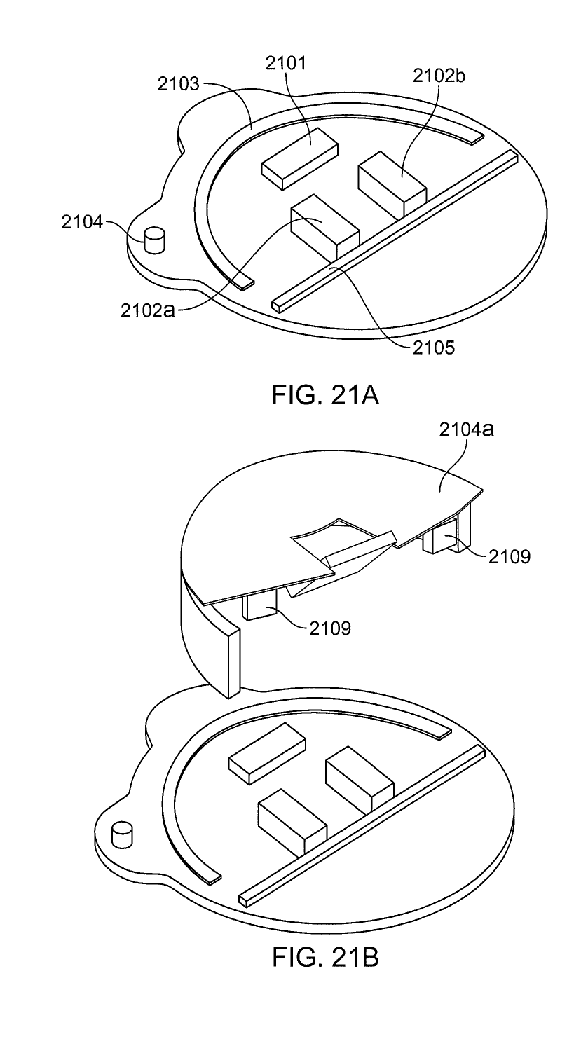

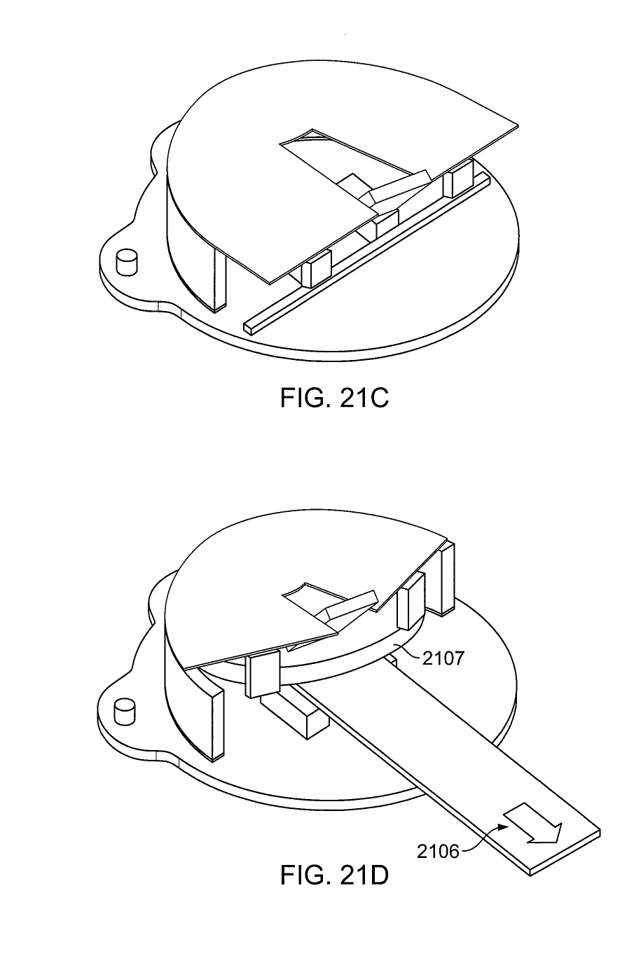

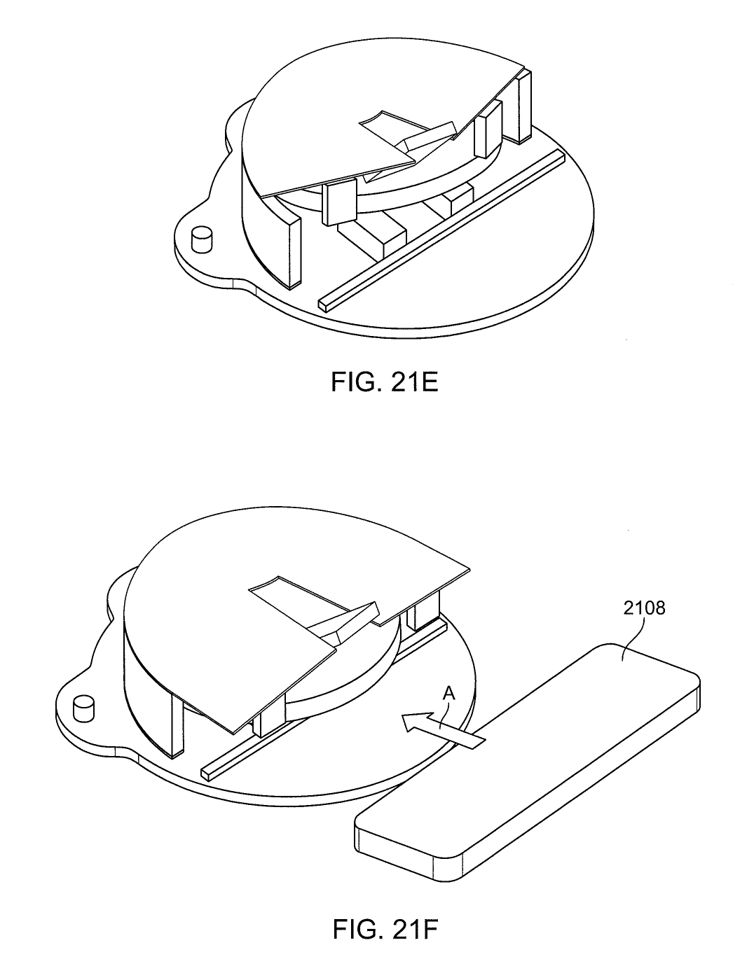

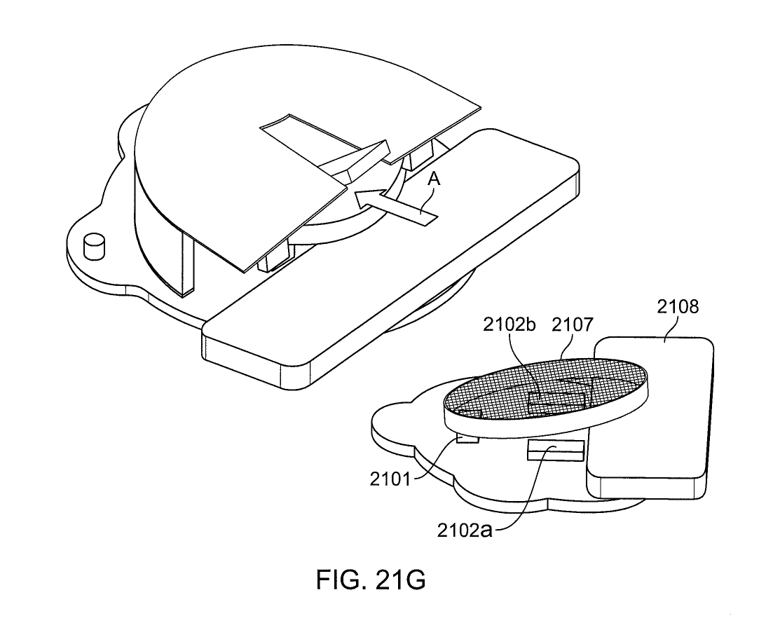

FIGS. 21A-E depict an assembling process of an On/OFF switch mechanism. As shown in FIG. 21A, first a battery positive terminal 2103, two battery negative terminals 2102a and 2102b, a LED 2104, a circuit boundary 2105, and a transistor fulcrum 2101 are mounted on the circuit board. In a typical implementation, the transistor fulcrum 2101 is lower than any of the negative battery terminals 2102a/2102b. Then as shown in FIGS. 21B-C, a metal battery retainer 2104a is aligned and soldered to the circuit board. The metal battery retainer 2104a has battery stoppers 2109 to maintain the battery. Then as shown in FIG. 21D, a pull tab 2106 is inserted to separate the connection to the battery, and subsequently the battery is inserted between the pull tab 2106 and the metal battery retainer 2104a. In some implementations, when a product manufactured in this way is dispatched from a factory, the pull tab 2106 is maintained as shown in FIG. 21D to prevent the power consumption due to the leak current between the battery and circuit board and also due to vibration disturbances that may try to wobble out the pull tab during transit. After the user pulls the pull tab 2106 out as shown in FIG. 21E, the circuit can be (or is) turned on, and the pull tab 2106 is no longer used.

FIGS. 21F-G depict an action mechanism for the On/Off switch during application. If the user wants to disconnect the circuit, he/she may push a key insert stick 2108 in a direction indicated by an arrow A, as shown in FIG. 21F. As shown in FIG. 21G, at the beginning the key insert stick 2108 is flushed with the metal battery retainer 2104a, and then slides inwardly along the direction indicated by the arrow A, typically very easily. As the key insert stick 2108 further advances in the direction indicated by arrow A, as shown in the partial cross-section in FIG. 21G, the battery is lifted above the battery negative terminals 2102a and 2102b, and thus the connection to the battery is blocked (i.e., the device is turned off). Conversely, the user may just pull out the key insert stick 2108 to turn the circuit switch on.

### Fourth Example

### Referred to Herein as “Model 220”

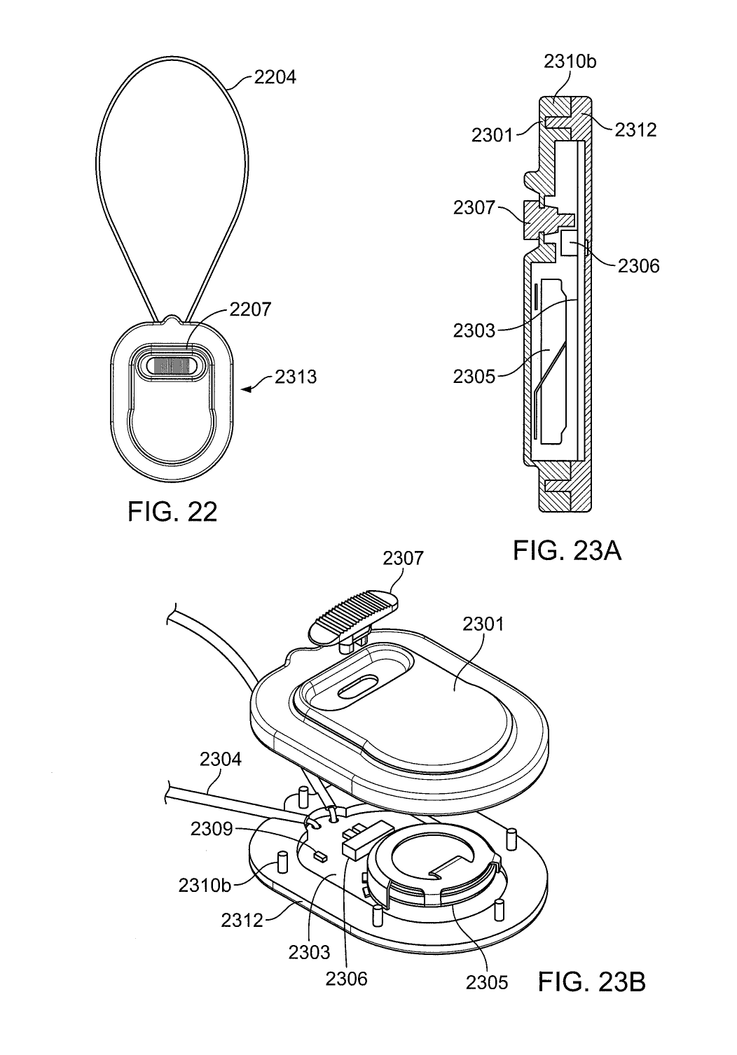

As shown in FIG. 22, a fourth example of the electromagnetic therapy device (Model 220) is constructed using a single wire loop antenna 2204, a finger slide switch actuator arm 2207, a plastic enclosure 2313, and electrical elements mounted in the plastic enclosure 2313.

FIGS. 23A-B depict a side cross-section view and an exploded view of the electromagnetic therapy device shown in FIG. 22. As shown in FIG. 23B, the plastic enclosure 2313 is comprised of a top plastic piece 2301 and a bottom plastic piece 2312, and these two pieces may be engaged together by press-fitting pins 2310b (2510b) in the bottom plastic piece 2312 into recessed sockets 2410a on the top plastic piece 2301. The setting and arrangement of the pins and recessed sockets as shown in FIGS. 23A-25 is only illustrative, and other setting and arrangement may also be used. For example, the pins may be distributed in both the top plastic piece 2301 and the bottom plastic piece 2312 to press-fit into corresponding recessed sockets distributed in both the bottom plastic piece 2312 and the top plastic piece 2301. Besides pins and recessed sockets, other mechanical engagement elements may also be used.

As shown in FIGS. 23A-B, the electrical elements, including a LED indicator 2309, a slide switch 2306, and a battery 2305, are mounted on a circuit board 2303 set on the bottom plastic piece 2312. Preferably, multiple batteries connected in series with disc battery insulator may be used to improve the power supply and to prevent the batteries from shorting to the metal retainer case, meanwhile keeping a compact size. The top plastic piece 2301 and bottom plastic piece 2312 may be transparent or opaque, but the area corresponding to the LED indicator 2309 thereof is preferably transparent to let the LED indicator 2309 illuminate through the same.

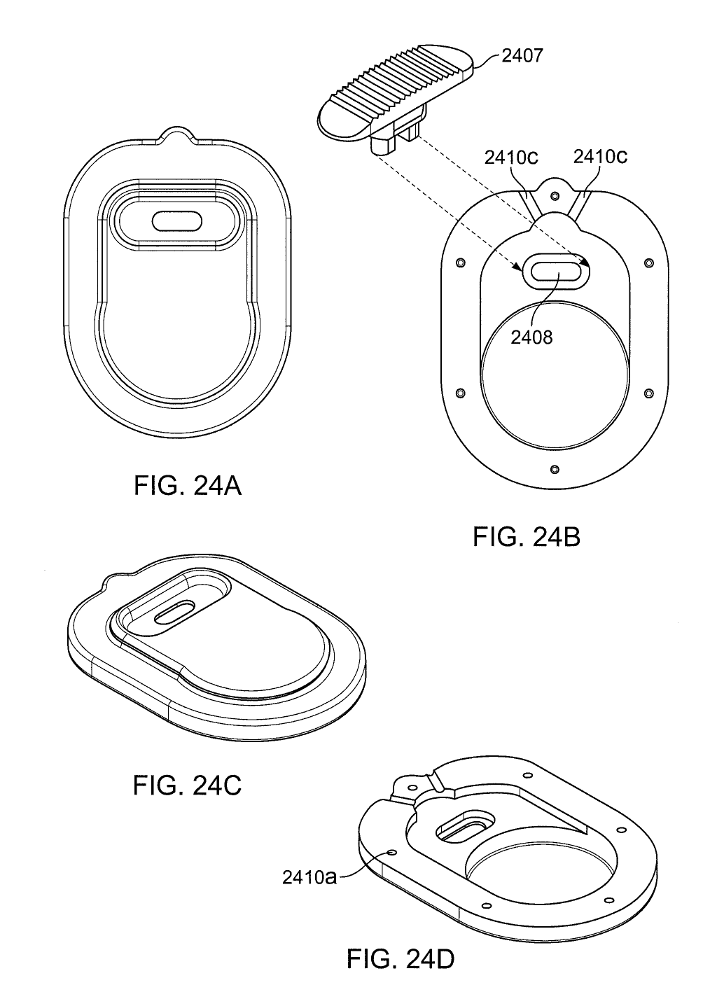

As shown in FIGS. 24A-D, the finger slide switch actuator arm 2407 gets pressed in to an open slot 2408 in the top plastic piece 2301 to electrically connect to the slide switch 2306. A user may push the finger slide switch actuator arm 2407 with a finger easily to activate the slide switch so as to activate the portable electromagnetic therapy device.

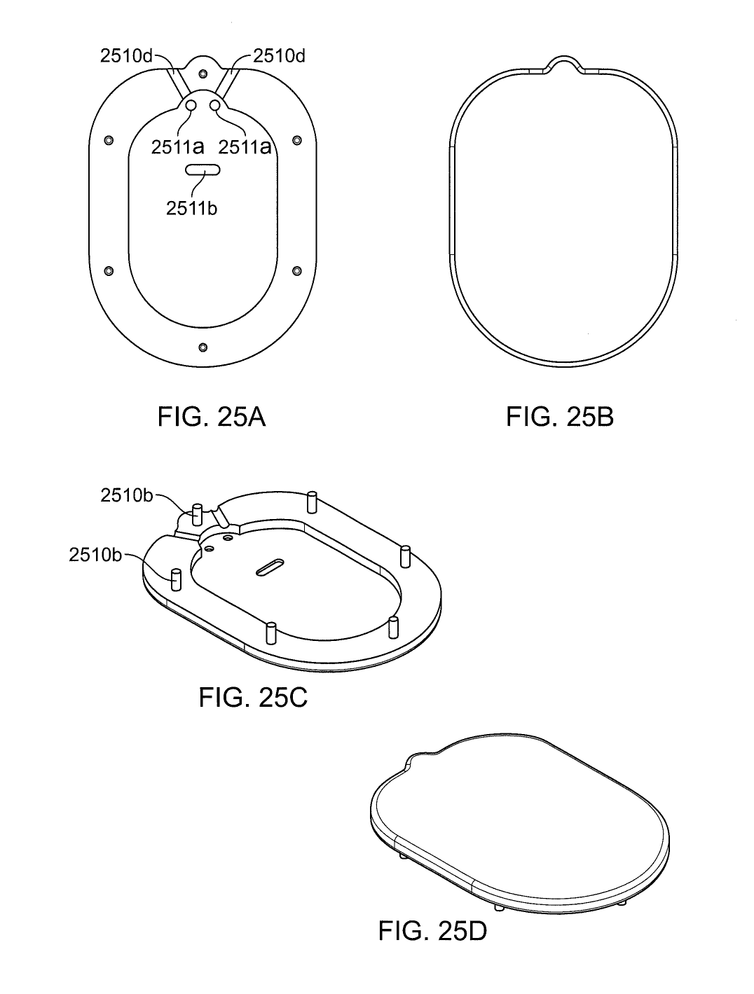

Preferably, there are corresponding channels 2410c (see FIG. 24B) and 2510d (see FIG. 25A) on the top plastic piece 2301 and the bottom plastic piece 2312 around which the single wire loop antenna 2304 can be tightly held and/or wound, so as to prevent the same from moving and damaged. As shown in FIG. 25A, on the surface of the bottom plastic piece 2312 where the circuit board 2303 is mounted, set antenna soldered shallow holes 2511a adjacent to the channel 2510d and switch shallow trough 2511b adjacent to the slide switch 2306 are formed, to help the circuit board 2303 to lay flat.

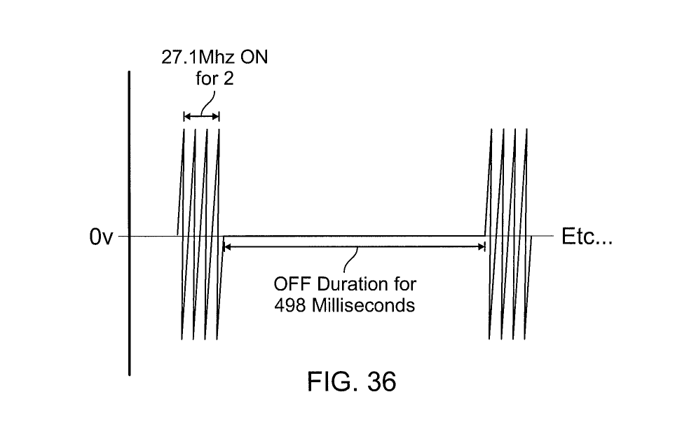

The Model 220 product typically offers a reduced size and lower manufacturing cost. The Model 220 electromagnetic therapy device can induce therapeutic properties by generating an RF output of 27.1 Mhz, being pulsed ON for 2 milliseconds and OFF for 498 milliseconds, as shown, for example, in the plot of FIG. 36.

### Fifth Example

### Referred to Herein as “Model 241”

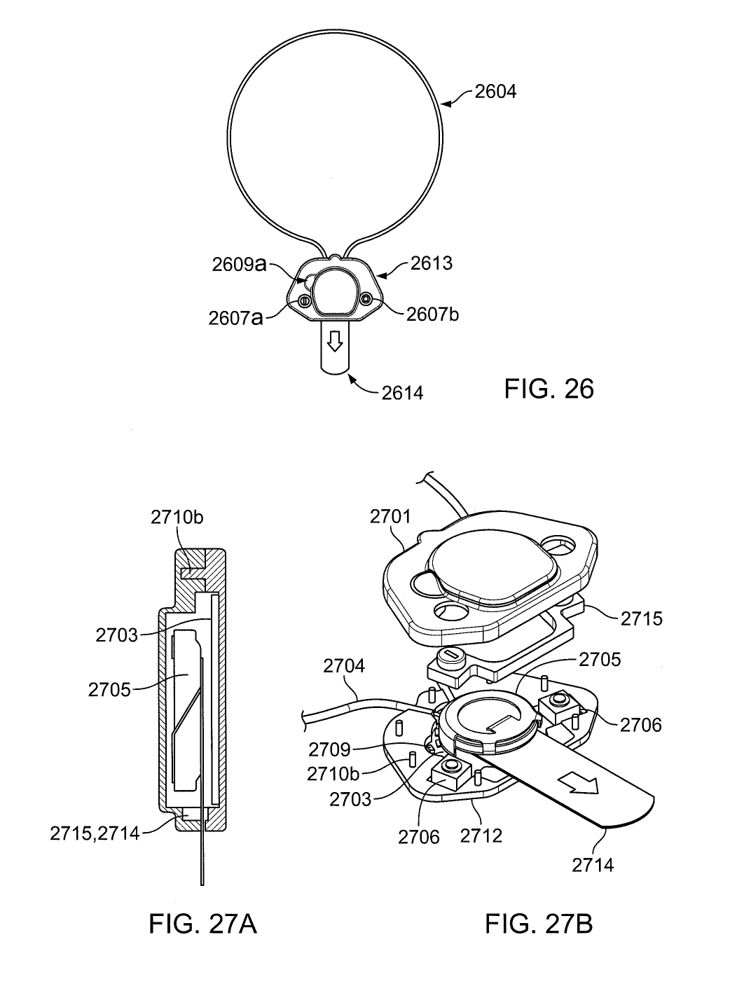

As shown in FIG. 26, a fifth example of an electromagnetic therapy device (Model 241) is constructed using a single wire loop antenna 2604, a momentary push button on switch 2607a and a momentary push button off switch 2607b, a LED dome lens compartment 2609a for containing a LED indicator, a pull tab 2614, a plastic enclosure 2613, and electrical elements mounted in the plastic enclosure 2613.

The LED dome lens compartment 2609a allows the LED indicator 2709 to illuminate through the same meanwhile helps the emitted light perceived by the user within a wider visual angle. For example, the LED indicator illuminates when the momentary push button on switch 2607a is pressed, and the LED indicator goes out when the momentary push button off switch 2607b is pressed.

FIGS. 27A-B depict a side cross-section view and an exploded view of a fifth example of a electromagnetic therapy device. As shown in FIG. 27B, the plastic enclosure 2613 is comprised of a top plastic piece 2701 and a bottom plastic piece 2712. The top and bottom plastic pieces 2701, 2712 may be engaged together by press-fitting pins 2710b (2910b) in the bottom plastic piece 2712 into recessed sockets 2810a on the top plastic piece 2701 (or vice versa). There is a rubber switch gasket 2715 inserted between the top plastic piece 2701 and the bottom plastic piece 2712, the area of the gasket 2715 is compressed when a pull tab 2714 is in place (inserted between a battery 2705 and a circuit board 2703, see FIG. 27B), so that the momentary push buttons cannot operate. As a contrast, when the pull tab 2714 is removed, then the momentary push buttons 2607a and 2607b are free to operate.

The setting and arrangement of the pins and recessed sockets as shown in FIGS. 27A-29 is only illustrative, and other setting and arrangement may also be used. For example, the pins may be distributed in both the top plastic piece 2701 and the bottom plastic piece 2712 to press-fit into corresponding recessed sockets distributed in both bottom plastic piece 2712 and top plastic piece 2701. Besides pins and recessed sockets, other mechanical engagement elements may also be used.

As shown in FIGS. 27A-B, the electrical elements, including a LED indicator 2709, push button switches 2706, and a battery 2705, are mounted on a circuit board 2703 set on the bottom plastic piece 2712. Preferably, multiple batteries connected in series with disc battery insulator may be used to improve the power supply meanwhile keeping a compact size. The top plastic piece 2701 and bottom plastic piece 2712 may be made of clear polycarbonate.

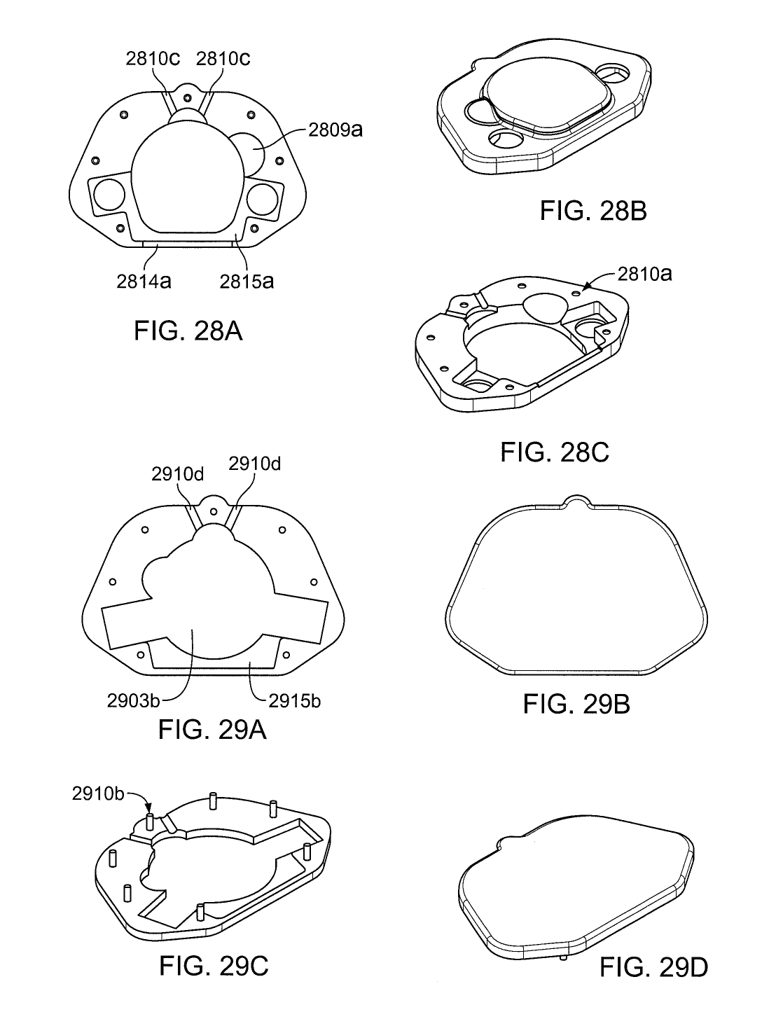

Preferably, there are corresponding channels 2810c (see FIG. 28A) and 2910d (see FIG. 29A) on the top plastic piece 2701 and the bottom plastic piece 2712 to be adjusted for tightly fitting around the single wire loop antenna 2704, so as to prevent the same from moving and damaged.

As shown in FIG. 28A, there is a pocket 2815a for the switch gasket and a pull tab slot 2814a on the bottom surface of the top plastic piece 2701. As shown in FIG. 29A, there is a pocket 2903b for the circuit board and a pocket 2915b for the switch gasket on the top surface of the bottom plastic piece 2712.

### Sixth Example

### Referred to Herein as “Model BW-001”

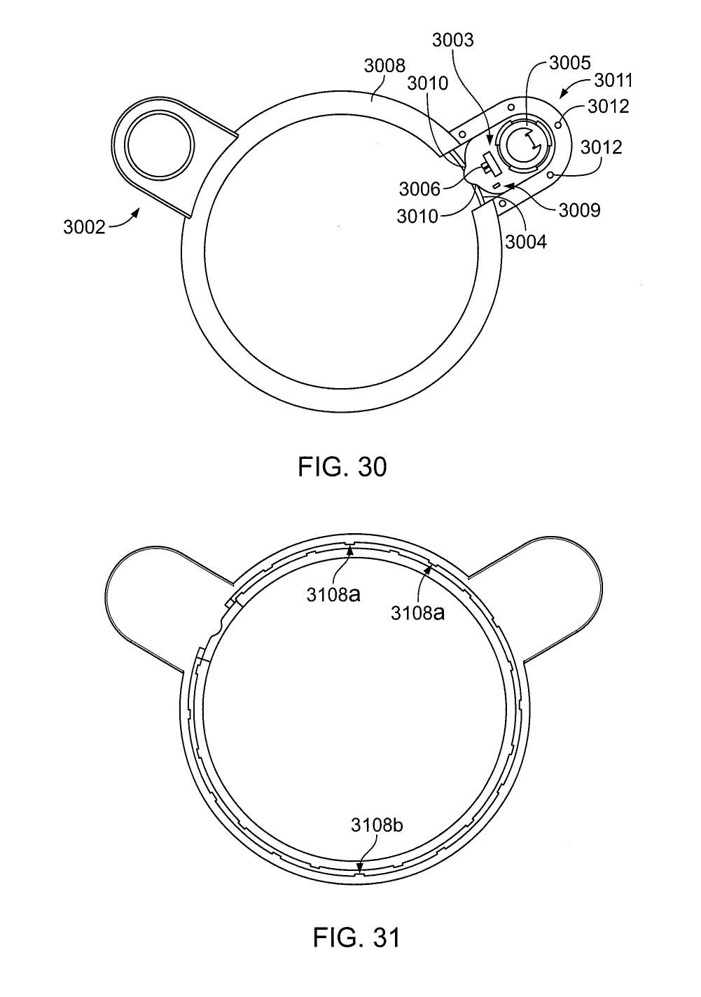

FIG. 30 depicts a top partial-cutaway view of a back wrap ring module, without a top housing, of a sixth example of an electromagnetic therapy device (Model BW-001). As shown in FIG. 30, the back wrap ring module is comprised of a dummy enclosure section 3002, a formed non-metallic ring 3008 for encapsulating and surrounding the single wire antenna 3004, and a PCB housing 3011 including a bottom housing post pins 3012 and a top housing (not shown). Different parts of the back wrap ring module may have different flexibility, for example, the dummy enclosure section 3002 may be slightly bendable, the ring 3008 may be soft and bendable, and the PCB housing 3011 may be non-bendable. The PCB housing 3011 is used for accommodating various electrical elements, such as a PCB 3003, a battery and metal retainer 3005, a PCB slide switch 3006, and a LED indicator 3009, wherein the single wire antenna 3004 is electrically connected to the PCB 3003 with single wire antenna connections 3010. Similar other implementations, the bottom housing post pins 3012 may be engaged with the top housing mechanisms such as post pins and corresponding recessed sockets.

As shown in FIG. 31, the ring 3008 may have prevention stubs 3108a to prevent the single wire antenna from coming out. Further, the ring 3008 may have trough structure 3108b, wherein the single wire antenna 3004 is press-fitted in place, so as to tightly encapsulating the antenna and prevent it from moving.

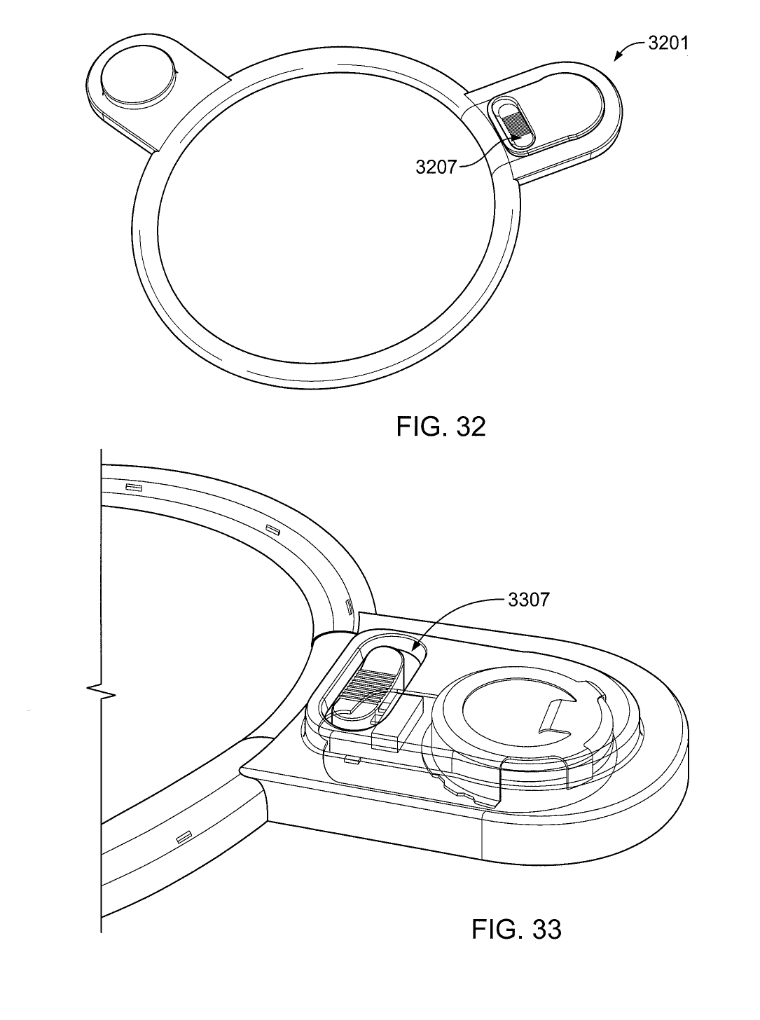

FIG. 32 depicts a top view of the fully assembled back wrap ring module of the sixth example, wherein a slide switch actuator arm 3207 is pressed into place, and the top housing 3201 is press-fitted over the bottom housing post pins 3012. Preferably, the top housing 3201, especially at least its part corresponding to the LED indicator 3009, is transparent so that the light emitted from the LED indicator 3009 may transmit through it and be perceived by the user. The LED indicator 3009 will illuminate when the slide switch actuator arm is actuated.

FIG. 33 depicts the internal details of the fully assembled back wrap ring module of the sixth example, wherein it is obvious that a slide switch actuator arm 3307 is within a shallow hole to help prevent against accidental exertion force disturbances, and that the PCB 3003 (see FIG. 30) is under the battery 3005 (see FIG. 30).

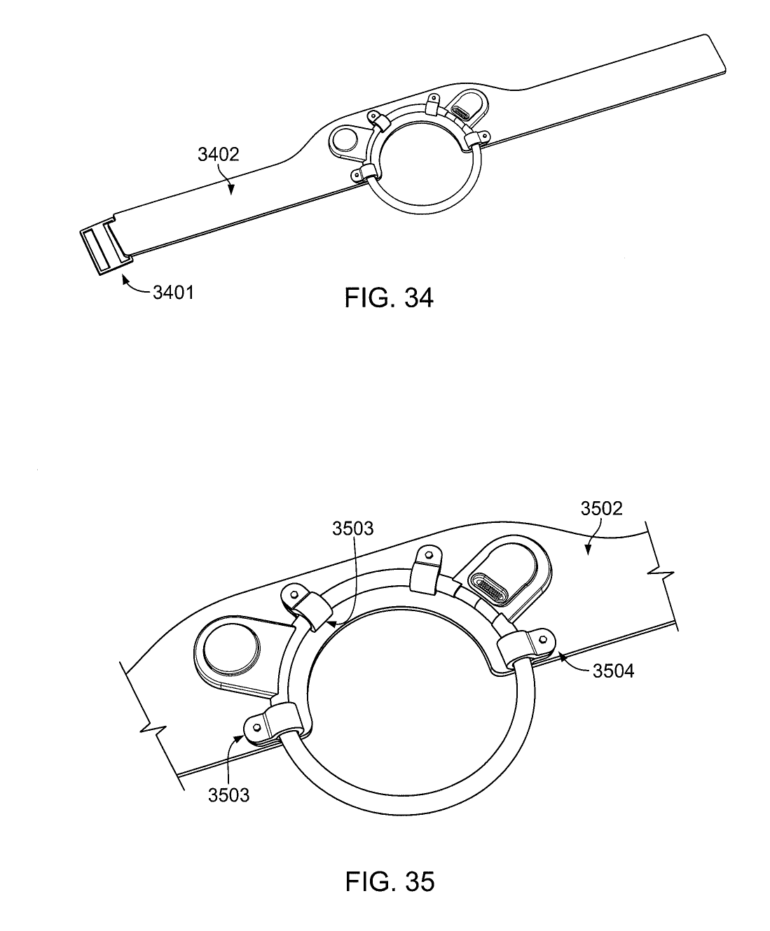

As shown in FIG. 34, when applied, the back wrap ring module of the sixth example is placed into a soft bendable material 3402 to be wrapped around a body to maintain comfortable constant treatment, and wherein one end of the soft bendable material 3402 may be provided with a buckle 3401 to hold the back wrap ring module to the body, while the other end of the soft bendable material 3402 is laced through the buckle end and is tightened as desired.

FIG. 35 shows a mechanism for holding the back wrap ring module in place, including straps 3503 and grommets 3504 mounted on the soft bendable material 3502 used for permanently holding the straps 3503 in place.

Single Loop Antenna

As shown in FIGS. 5A-I, the antenna may have various configurations. In some implementations, the antenna can be a sing loop wire, as shown in FIGS. 11A-C. Although the antennas shown in FIGS. 11A-C have symmetrical shapes, asymmetrical shapes may also be used. For example, the symmetrical shapes may be selected from a group consisted of a circle, an ellipse, and a rectangle.

Further, the antenna 1104 may be set on one side of the circuit board 1102, as shown in FIGS. 11A-B (Model 071 and Model 077/078). But as shown in FIG. 11C, the antenna 1104 may be reversed to the other side of the circuit board 1102 (Model 150).

The single loop wire has a length depending on the body site where the portable electromagnetic therapy device is applied and its characteristics including thickness, resistance, and material. For example, changing the wire characteristics such as thickness or resistance of the wire will allow the length to increase or decrease. Moreover, the conductor material of the single loop wire may be tin or gold, and if different material is applied for the wire, different lengths are required. For example, if the single loop wire has a diameter thickness of 0.8128 mm or 20 gauge, is circle-shaped, and is made of low resistance copper metal, preferably its length ranges from 3.14 mm-47.12 cm.

Various implementations may have antenna 1104 of different lengths. For example, Model 077/078 as shown in FIG. 11B has a longer antenna 1104 compared to that of Model 071 as shown in FIG. 11A, while the antenna 1104 of Model 150 as shown in FIG. 11C has the same length as that of Model 071 as shown in FIG. 11A.

Besides, the antenna 1104 is bendable to conform to the body curves of the body site where the portable electromagnetic therapy device is applied. In order to protect the naked antenna 1104 from polluted or damaged by the external environment, preferably the antenna 1104 is tightly encapsulated by an injection molded ring. Meanwhile the injection molded ring may be a semi-rigid ring to maintain the flexibility to better adapt to the body curves of the body site where the portable electromagnetic therapy device is applied.

Moreover, preferably the portable electromagnetic therapy device is not directly in contact with the body site where it is applied, since the body site may perspire and the sweat may pollute and erode the device on contacting the same. In some implementations, there may be a gel pad between the portable electromagnetic therapy device and the body site to both prevent the body fluid from penetrating to the device, to protect the device against excessive force, and, in some implementations, to maintain good air permeability for the skin. In some implementations, the circuit elements besides the antenna may be sealed in a hardened moisture resistant enclosure 1108 (see FIG. 11A) so as to protect the inside electrical elements from moisture of the external environment. In order to make the user comfortable, preferably the thin film layer substrate 1712, which may directly contact the user's skin, is made of a soft fabric and/or foam or other hygroscopic material to absorb the moisture and sweat from the skin.

In Models 071, 077/078, 150, 088, 240, the activator 1006 is implemented with one or more mechanisms, such as pull tabs, dome switches, shorting pads, key insert sticks, but these implementing manners are only illustrative, and the activator 1006 may apply one of a press switch assembly, a slide switch assembly, and a tactile press switch assembly.

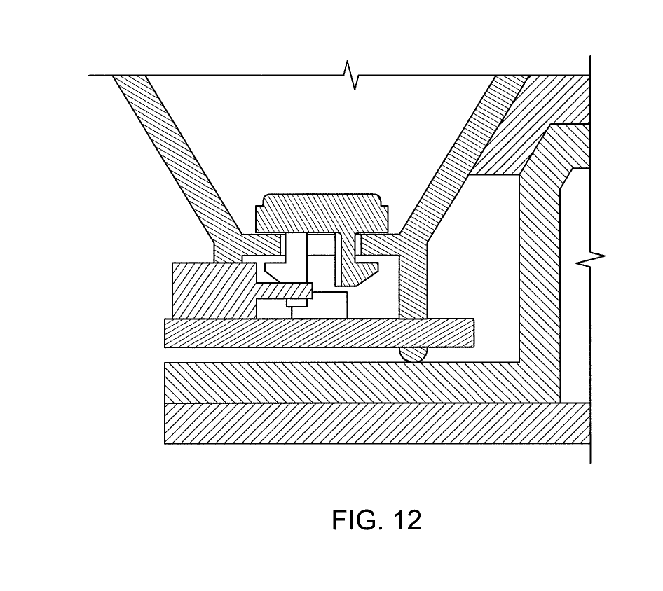

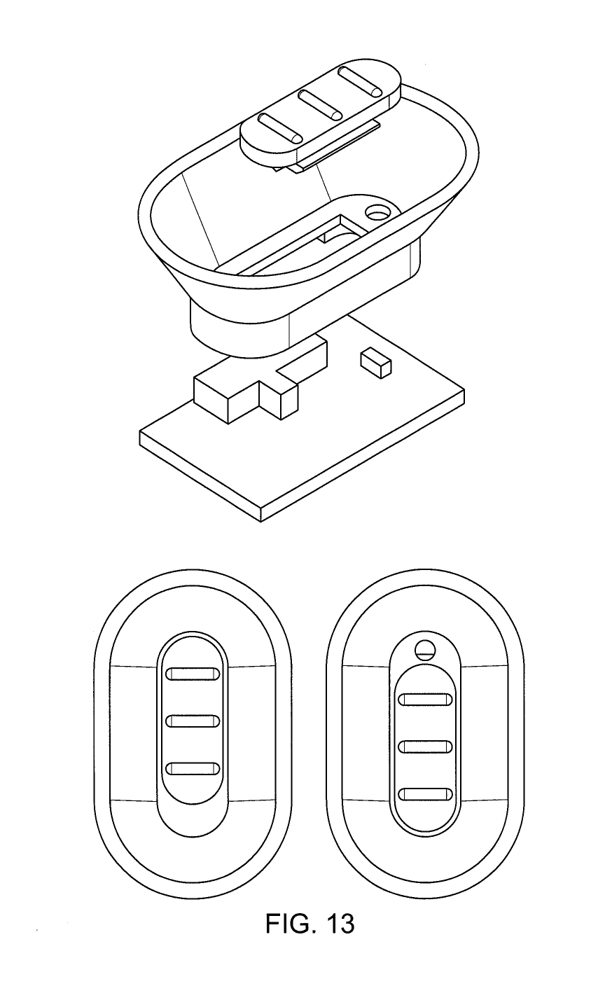

FIGS. 12-13 show examples of the slide switch assembly, and it is constructed by stacking an injection molded switch channel, an injection molded switch cover, and a slide switch set on the circuit board. Further, since the portable electromagnetic therapy device often vibrates with the human body where it is applied or if the device rubs up against an external object, various means are applied to protect the slide switch assembly from accidental activation, for example, as shown in FIG. 12, there may be a button clearance between the top surface of the injection molded switch channel and the top surface of the injection molded switch cover so that the injection molded switch cover will not be easily touched and pushed to bring about an accidental activation. Generally, the button clearance is 0.05 mm to 25.4 mm, depending on the enclosure structure wherein the switch should fit correctly.

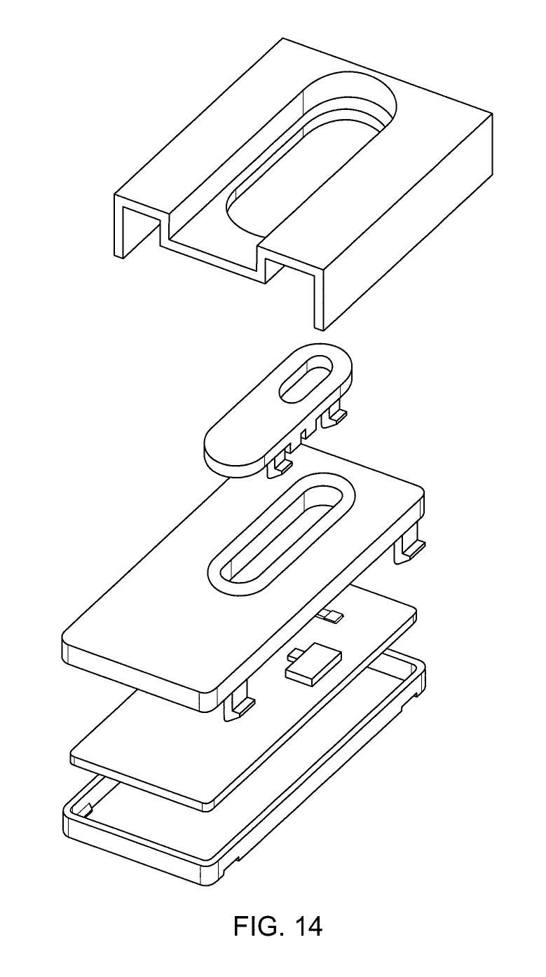

FIG. 14 shows another example of the slide switch assembly, and it is constructed by stacking an injection molded thermoplastic elastomer outer shell, an injection molded button, an injection molded top cover, and a slide switch set on the circuit board.

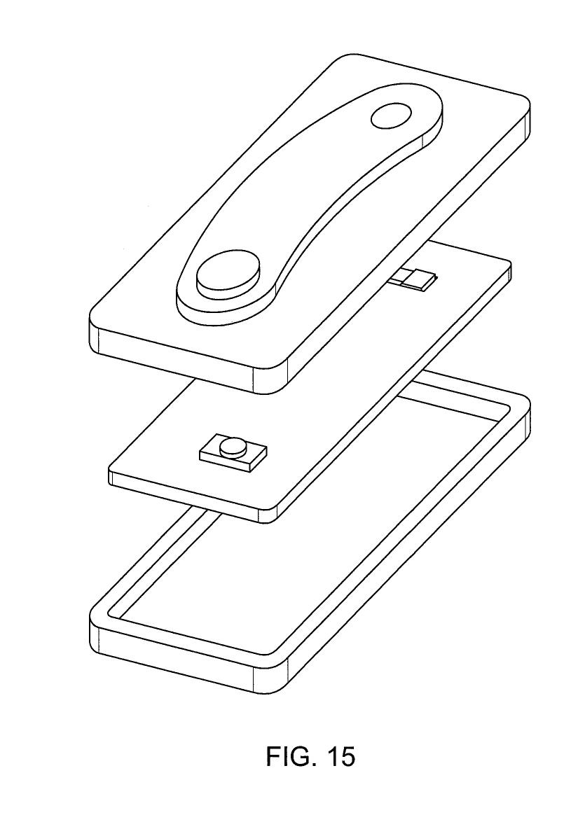

FIG. 15 shows an example of the tactile press switch assembly, and it is constructed by stacking a molded silicone rubber or injection molded thermoplastic elastomer outer shell, a momentary switch set on the circuit board, and a molded silicone rubber or injection molded thermoplastic elastomer bottom shell.

The above examples illustrate several possible constructions of the electromagnetic therapy device and do not limit its design or construction.

In some implementations, the indicator as mentioned above indicates the status of the portable electromagnetic therapy device, such as turned on or off.

In some implementations, the indicator is a light-emitting diode, which transmits different lights depending on the status of the portable electromagnetic therapy device. Preferably, the lights are visible (including white light) (from Far infrared to Ultraviolet or red color in nature to a purple color in nature, a typical human eye will respond to wavelengths from about 390 nano meter to 750 nano meter), and render different colors depending on the status of the portable electromagnetic therapy device, so that the user may determine the operating status according to the colors of the visible lights with their own eyes.

Of course, the lights may either be invisible (Infrared or ultraviolet range) but need a corresponding sensor to pick it up and further processing to inform the user of the current operating status of the portable electromagnetic therapy device.

In some implementations, the portable electromagnetic therapy device includes a treatment timer, and the light-emitting diode changes its luminosity as the timing of the treatment timer lapses. It is also applicable that as the battery decays the light-emitting diode changes its luminosity without using a treatment timer.

### Methods of Using Pulsed Electromagnetic Field (PEMF) Therapy in Treating Certain Diseases

Bone and Joint Disorders:

The urine of patients with bone and joint disorders typically shows elevated levels of hydroxyproline, hexosamine, creatinine, and uronic acid as a result of metabolic errors in connective tissues surrounding the affected site. Not only can these errors be corrected with PEMF therapy, but also joint pain and swelling can be reduced and mobility of the joint increased. Another major advantage of PEMF therapy is that it significantly reduces the time required to heal fractured bones. It has also proven to be effective for osteomyelitis, osteoarthritis, rheumatoid arthritis, cervical spondylosis, and lower back pain (including that caused by disc displacement).

Diabetes Mellitus:

Blood sugar levels may be slowly reduced to normal or near normal with application of a pulsed electromagnetic field (PEMF). Although the mechanism of action is not completely understood, the evidence obtained thus far indicates that the procedure not only increases the metabolism of glucose in the tissues but also increases the production of insulin and enhances insulin binding to its specific receptors. The therapy has also proven to be effective for gastritis, peptic ulcer, ulcerative colitis, irritable colon, and hemorrhoids.

Bronchial Asthma:

Bronchiolar obstruction can be gradually reduced with PEMF treatment, which liquefies the mucous and facilitates spontaneous clearance. PEMF therapy also has anti-inflammatory action, which helps to ensure that the airways remain free and functional. In patients who have undergone the treatment, Forced Vital Capacity, Forced Expiratory Volume, and Peak Expiratory Flow Rates have increased and wheezing and dyspnea have significantly improved. The treatment is also effective for the common cold, tonsillitis, sinusitis, chronic bronchitis, and bronchiectasis

Cardiovascular Diseases:

PEMF therapy is useful in the prevention of heart attacks in hypertensive patients. Treatment helps to lower blood cholesterol levels and increase the circulation of blood by centrally mediating vascular dilatation. This is particularly important in preventing platelet aggregation and maintaining adequate oxygenation and nutrition of cardiovascular and other tissues. PEMF therapy also effectively disintegrates atherosclerotic plaques. An additional advantage of the procedure is that it blocks the production of free radicals, which play a major role in cardiovascular damage at the cellular level. Other vascular conditions for which PEMF may be effective are phlebitis, endarteritis, and varicose vein.

Brain and Mind Disorders:

Directed through the skull at different points, the PEMF can, by inductive coupling, produce an electric current in specific areas of the brain. It may thus be possible to enhance higher brain functions such as learning, memory, and creative thinking by selective stimulation of certain cells. PEMF may have broad application as the modality of choice for psychological disorders such as depression, aggression, anxiety, and stress as well as for Parkinson's disease, epilepsy, migraine, stroke, Alzheimer's and other degenerative brain disorders. In addition, it is believed that cerebral palsy, mental retardation, hyperactivity, learning disabilities may be improved by PEMF stimulation of the central nervous system.

PEMF therapy can increase the efficiency of brain cells in synthesizing the neuro-chemicals required for the transmission of impulses or commands at the synaptic level and by improving the electrical activity of these cells. The brain is a neuro-chemical complex. The efficiency of the brain or intellectual capacity of the brain depends upon the efficient performance of the brain cells and production of the chemicals that are called neurotransmitters.

Too much dopamine can result in hyperactivity, while too little can result in uncoordinated movements of the limbs (Parkinsonism). Less acetylcholine, a neuro-chemical, in the brain is a reason for dementia especially of the Alzheimer's type. If the brain cells are stimulated repeatedly, after showing inhibition, they rebound and become more active than prior to stimulation. Since PEMF has the ability to stabilize the genes and prevent the activity of oxygen free radicals formed in the cells, it helps to retard the aging process.

Genitourinary Conditions:

PEMF has been successfully used to treat genitourinary conditions such as menstrual irregularity, sterility, endometritis, and endometriosis in women and orchitis, prostatitis, and oligospermia in men.

Preoperative and Prophylactic Therapy:

PEMF therapy over the epigastrium can provide increased blood profusion to the body's extremities to reduce the inflammatory response to injury. Preoperative treatment of the surgical site has also been shown to accelerate healing.

Post-Operative Recovery:

PEMF or TENS over 1.5 inches above the wrist line may reduce or ease the nausea for post-surgical recovery, motion sickness or other forms of nausea symptoms such as vomiting.

Non-Contacting Induction of Electrical Current in Tissue

Devices described herein can induce current at a high frequency. The amount of current induced by a device is partly proportional to the frequency. Modulating a carrier waveform, such as the pulse modulation of 27+/0.5 Mhz (e.g., 27.1 MHz) in devices described herein, allows a larger current to be produced in a tissue than the pulse modulation waveform alone. The pulse modulation is selected for time and amplitude characteristics appropriate to biological systems. The carrier wave ensures that induced current has a magnitude that is maintained coherently within the pulse modulation. A varying pulse modulation is sustained by a similar magnitude of induced current. Rectification occurring in biological systems, such as across cellular membranes, causes the originating pulse modulation waveform to appear as a low frequency voltage. Membrane capacitance allows induced currents to enter cells much more easily than the pulse modulation waveform would by itself. Shunting of current around cells rather than through the cells is also reduced.

No conductive contact of the device with the tissue is required to induce the electrical current in the tissue. The size of the antenna of the device, being much smaller than a wavelength, ensures that the emission is localized to the treatment area. Accordingly, there is generally little far-field emission that might interfere with, for example, domestic appliances.

The devices described herein generally induce current at a much higher frequency than tissue-stimulating devices such as, for example, inductive bone-healing stimulators that pulse coils to produce a magnetic field or capacitive stimulators that produce a pulsed electric field.

Positioning of Therapeutic Devices

Therapeutic devices such as a PEMF apparatus, a transcutaneous electrical neural stimulator (TENS), or a static magnet array can be positioned at particular points on the body to achieve an enhanced medical therapeutic effect, e.g., accelerate healing, reduce pain, swelling and bruising. TENS operates by causing an electric current to be passed between electrodes placed on the skin over, for example, a painful area. Devices are described herein that can induce electrical current in a bodily tissue without the use of electrodes that are applied to the skin.

In some implementations, the electromagnetic therapy device can be placed over clothing. For example, the electromagnetic therapy device can, in some implementations, be placed in a shoe as part of an insole. The device cam generate therapeutic electromagnetic waves that aid in reducing symptoms, such as pain or swelling in a person's foot. If there is fabric between the foot and the device, such as a sock, the electromagnetic waves emitted from the device can penetrate through the fabric to the point of injury in the patient.

In some implementations, a therapeutic device can be positioned and operated at a specific acupuncture point, including but not limited to the following: the external end of the elbow transverse crease; the depression at the lower border of the malleolus lateralis; below (e.g., about 1 inch below) the lateral extremity of the clavicle at the level of the first intercostals space; between the fourth lumbar vertebra and the fifth lumbar vertebra; 1 inch to the right or left (horizontally) of the position between the fourth lumbar vertebra and the fifth lumbar vertebra; a depression anterior or inferior to the head of the fibula; about 1.5 inches above the medial border of the patella; between the radius and the palmaris longus; or at a position of pain (e.g., where the pain sensation is the strongest in an individual).

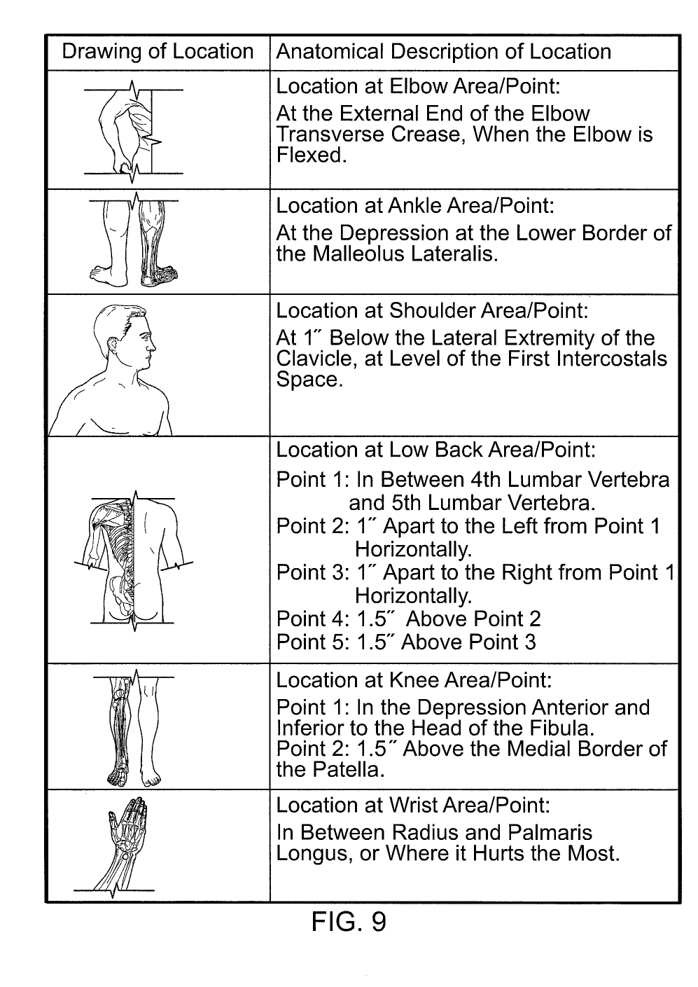

FIG. 9 depicts example anatomical locations where a therapeutic device described herein can be placed on an individual as part of a treatment program (e.g., a treatment for the reduction or elimination of pain).

The therapeutic devices described herein can be used in combination with specific acupuncture positioning techniques to reduce or eliminate pain. Examples of pain-related disorders include, for example, pain response elicited during tissue injury (e.g., inflammation, infection, and ischemia), pain associated with musculoskeletal disorders (e.g., joint pain such as that associated with arthritis, toothache, and headaches), pain associated with surgery, pain related to irritable bowel syndrome, and chest pain.

Insole Electromagnetic Therapy Device for Treatment of Foot Injury

In some implementations, the electromagnetic therapy device can be positioned beneath or adjacent to a patient's foot to treat an injury in or on the foot. For injuries near the heel or sole of a foot, however, there are multiple issues that make it difficult to maintain the position of the device during treatment. For example, adhering the device to the bottom of a patient's foot may make walking uncomfortable. In addition, if a patient were to walk or run with the device located near the sole of the foot, the pressure applied to the device during each step could cause significant damage to the device. If the electromagnetic therapy device is formed as part of or within a resilient insole, however, the discomfort associated with the device near the foot can, in some implementations, be avoided. In addition, in some implementations, the resilient insole material can protect the electromagnetic therapy device from damage caused by the downward foot pressure associated with walking or running.

As an example, the electromagnetic therapy device can be encapsulated in resilient material that is capable of fitting into a shoe as an insole. The resilient material can be composed of any suitable material that protects the electromagnetic therapy device from excessive pressure and preferably does not cause discomfort to the patient. For example, the resilient material can be composed of a rubber or a gel material. A suitable gel-like material includes the gel used for Dr. Scholl's insoles. The gel itself can be encased in a thin and flexible plastic cover.

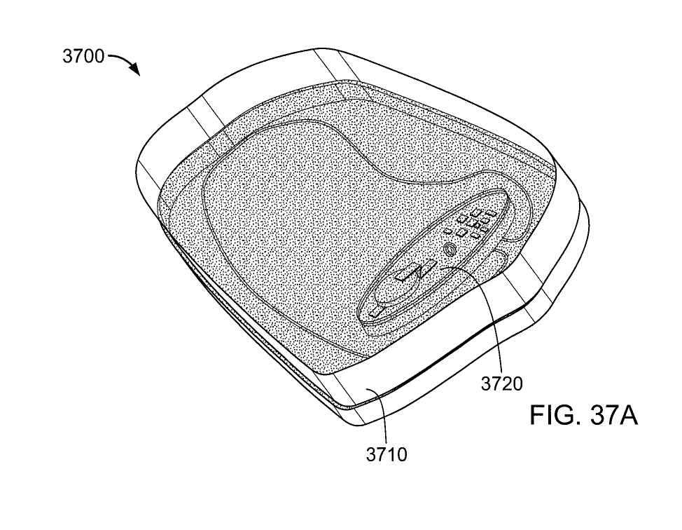

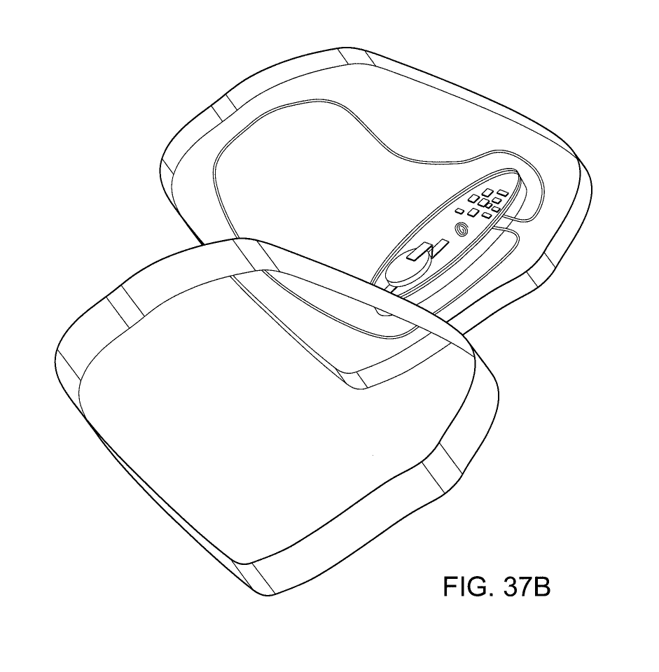

FIG. 37A is a schematic of an example insole electromagnetic therapy device 3700 that includes a therapeutic electromagnetic device 3720 encapsulated in a gel material 3710. The size of the gel material 3710 can be made to match the insole of a shoe (e.g., the insole of a dress shoe, running shoe, sneaker or sandal). The insole 3700 composed of the gel material 3710 and the device 3720 can be designed to have other sizes as well. For example, the gel material 3710 shown in FIG. 37A has a width of about 58 mm and a length of about 100 mm. The thickness of the gel material 3710 can be constant over the length of the device 3700. Alternatively, the thickness of the gel material 3710 can vary. For example, the thickness can be about 7.5 mm or more (e.g., 10 mm) near the portion of the device 3700 that is to be aligned with a patient's heel. Nearer to a portion of the device 3700 that is adjacent to a patient's toes, however, the thickness of the gel material 3710 can be reduced to about 6 mm or less.

A process for fabricating the device 3700 can include, for example, placing the therapeutic electromagnetic device 3720 between two separate pieces of gel material, each about 3 mm or less in thickness, and bonding the pieces of gel material together using a suitable adhesive. For example, FIG. 37B is a schematic of an example of an insole electromagnetic therapy device, in which a top gel encasing can be separated from a bottom gel encasing, thus allowing a user access to the electromagnetic therapy device if for example, the user wants to replace the device.

As in other implementations, the therapeutic electromagnetic device 3720 can be activated using a preserver circuit, which implements the ON/OFF function of the device 3720 by electromagnetic induction. For example, it may be useful in some implementations to test operation of the device 3720 after the device 3720 has been placed between the pieces of gel material. Alternatively, it may be desired to activate the device 3720 remotely because the gel material prevents manual access to the electromagnetic device circuit. Because it operates by electromagnetic induction, the preserver circuit enables remote activation without requiring manual access to the device 3720. The preserver circuit also can be referred to as a “piercer” circuit.

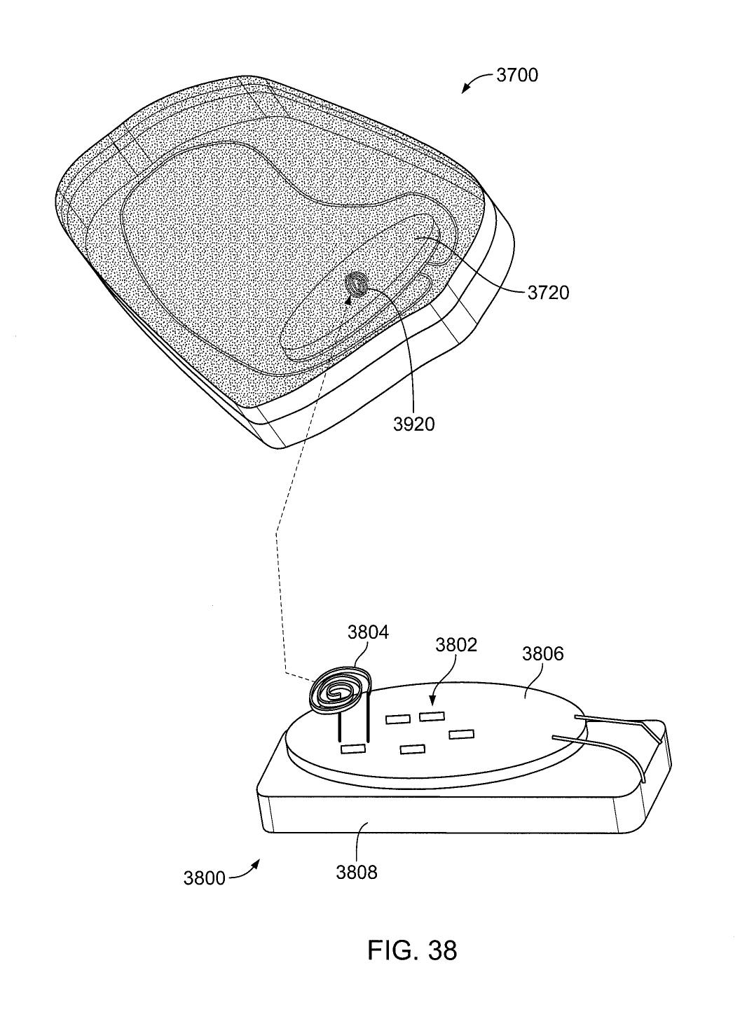

FIG. 38 is a schematic of an example preserver circuit 3800 for implementing the ON/OFF function of a therapeutic electromagnetic device 3720 that is located between gel material for forming an insole. The preserver circuit 3800 produces an 8 MHz frequency pulse that can turn on or shut off the therapeutic electromagnetic device 3720 of an insole electromagnetic therapy device 3700. The preserver circuit 3800 includes circuit components 3802 and coil 3804 formed on a printed circuit board 3806. A battery 3808 drives the preserver circuit 3800. The coil 3804 serves as an antenna to emit the electromagnetic pulse to a corresponding coil 3920 of a receiving circuit on the therapeutic electromagnetic device 3720. Upon receiving the electromagnetic pulse, the therapeutic electromagnetic device 3720 can be turned on or off.

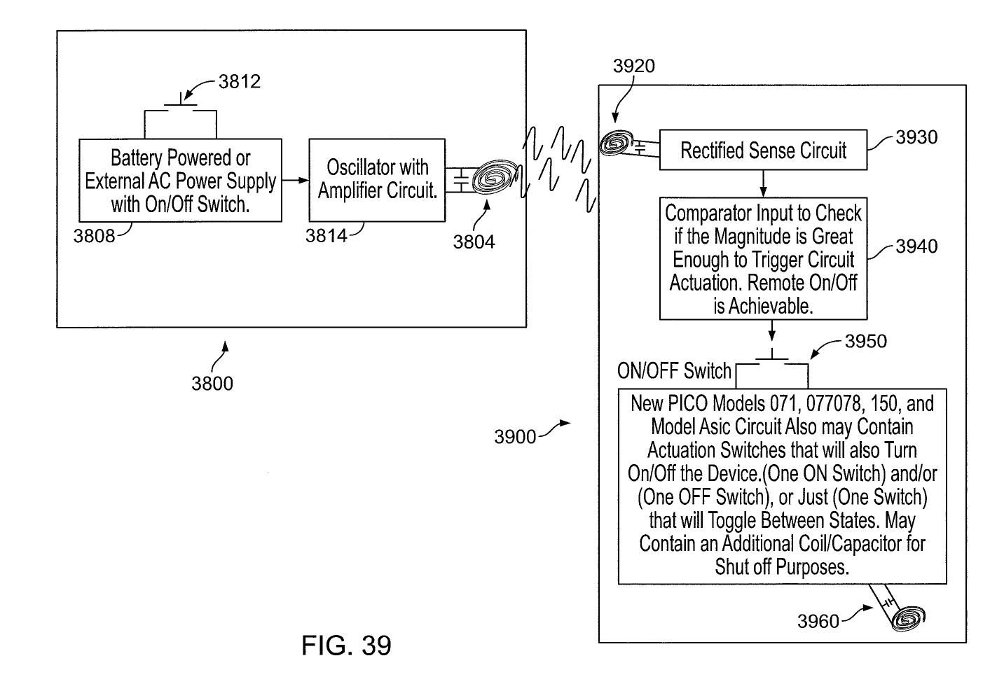

FIG. 39 is a circuit diagram schematic of a preserver circuit 3800 and a corresponding receiving circuit 3900 formed on the therapeutic electromagnetic device 3720. As shown in FIG. 39, the preserver circuit 3800 includes a battery 3808, a switch 3812 (e.g., a push-button switch) coupled to the battery, an oscillator circuit 3814 coupled to the switch 3812 and battery 3808, and a blast coil 3804 coupled to the oscillator circuit 3814.

The receiving circuit 3900 includes a coil 3920 coupled to a rectifying circuit 3930, a comparator 3940 coupled to the rectifying circuit, and a switch 3950 coupled to the comparator 3940. Various switches can be used. For example, the switch 3950 can be a single action switch, in which activation of the switch only turns the device ON or only turns the device OFF. Alternatively, the switch can include a toggle action, in which activation of the switch turns the device ON or OFF. In some implementations, the switch can include a second coil 3960 that allows wireless de-activation of the device using electromagnetic induction.

During operation, a user activates the switch 3812 of the preserver circuit, for example, by depressing a push-button switch. Activation of the switch 3812 transfers enables a voltage to be transferred from the battery 3808 to the oscillator circuit 3814. For a push-button switch 3812, activation can include momentarily depressing the push-button. The oscillator circuit 3814 transforms the voltage signal into a pulse signal, which is amplified by an amplifier circuit included in the oscillator 3814. The frequency of the pulse can be tuned as is known in the art using a capacitor that is in either series or parallel resonance with the oscillator circuit and the coil 3804. The amplified signal then is passed to the coil 3804, which emits a signal intended for the receiving circuit 3900. An example signal produced by the preserver circuit 3800 can have a duty cycle on time of about 200 microseconds and an off time of about 1 ms. This provides 20% of the on time to the coil 3804 so that it is not necessary to repeatedly push the switch 3812 to activate the circuit.

The coil 3920 on the receiving circuit 3900 receives the signal emitted from the coil 3804 and transmits the received signal to the rectifying circuit 3930. The rectifying circuit 3930 is tuned to the same frequency as the oscillator circuit in order to detect the wireless signal. The rectifying circuit 3930 converts the signal to a magnitude and provides the rectified signal to the comparator 3940. If the magnitude is above a pre-defined threshold set by the comparator 3940, the comparator 3940 activates the switch 3950 so that the device 3720 turns ON or OFF, depending on the switch used.

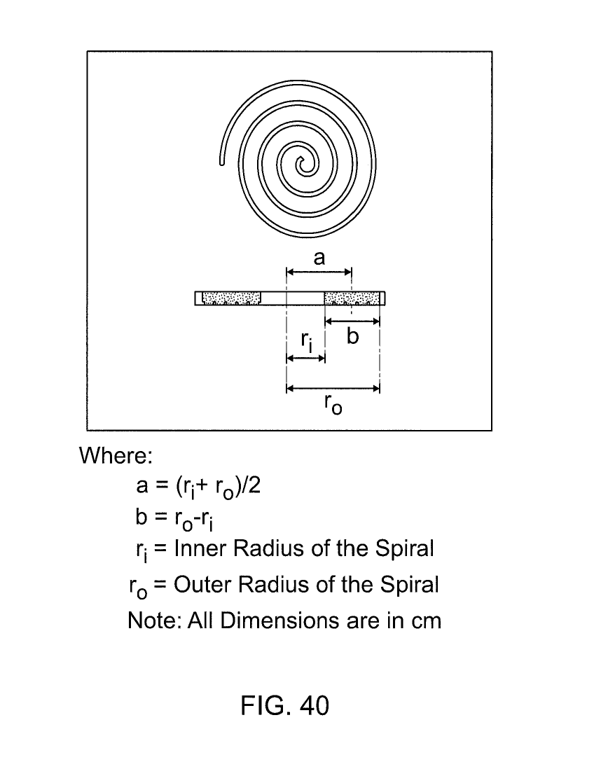

As explained above, the preserver circuit 3800 including the coil 3804 is tuned with a capacitor. The resonant frequency can be expressed as f=1/(2π\sqrt{LC}), where L is the inductance of the coil 3804 and C is the capacitance of the capacitor. The coil 3804 may be a wound coil or a coil formed on a printed circuit board. For example, the coil 3804 can be composed of an 18 gauge solid enamel wire shaped into a pancake inductor coil. FIG. 40 is a schematic of an example of a pancake inductor coil. The inductance of the pancake inductor coil can be expressed as L=(0.3937)(aN)²/(8a+11b), where a is the average of the inner radius rᵢ of the spiral and the outer radius rₒ of the spiral, b is the difference between the inner radius rᵢ and the outer radius rₒ, and N is the number of turns of the coil. The pancake inductor coil can be formed on or a part of the printed circuit board and help reduce manufacturing cost and minimize package size. For example, the coil can have an outer spiral radius of about 4 mm.

The voltage generated in the receiving coil can depend on the mutual inductance between the two coils, which is a function of coil geometry and the spacing between the coils. The induced voltage is proportional to 1/x³, where x is the distance between the coils. The voltage generated in the receiving coil can be expressed as V=−M di/dt, where di/dt is the change in current with time in the first coil, and M is the mutual inductance. M can be expressed as

$$M = [ \frac{{μ}_{0} π {N}_{1} {{N}_{2} ( {r}^{1} {r}^{2} )}^{2}}{2 {( {r}_{1}^{2} + {x}^{2} )}^{3 / 2}} ] ,$$

where μ₀ is the magnetic permeability of the coils, N₁ is the number of turns of the coil in the preserver circuit, N₂ is the number of turns of the coil in the receiving circuit, r₁ is the radius of the coil in the preserver circuit, and r₂ is the radius of the coil in the receiving circuit

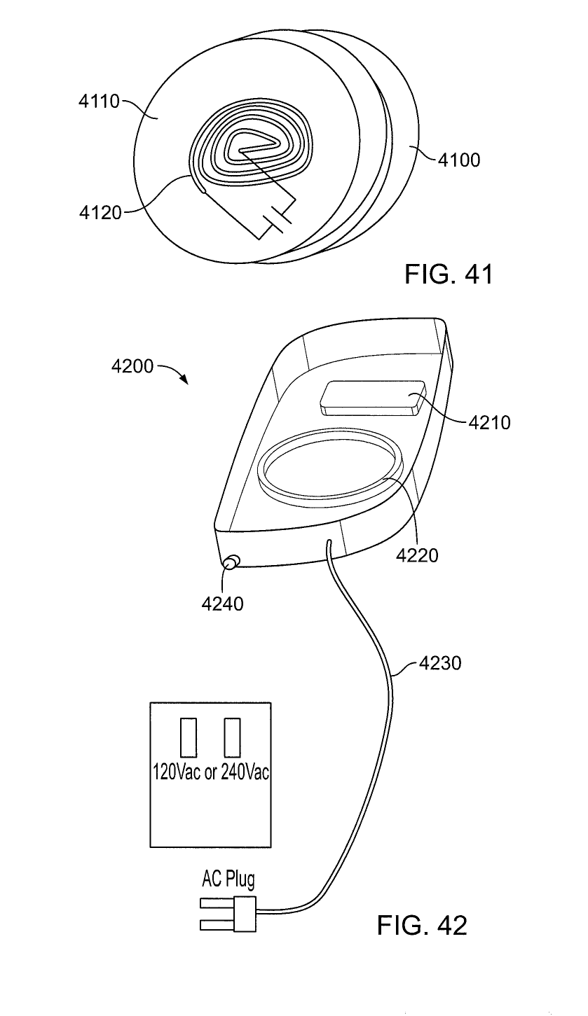

In some implementations, the therapeutic electromagnetic device includes a metal film or sheet positioned near the coil to help shield the coil from stray electromagnetic fields. FIG. 41 is a schematic of an example printed circuit board on which a pancake coil is formed. The remaining portions of the receiving circuit of the therapeutic electromagnetic device are excluded from the schematic for ease of viewing. As shown in FIG. 41, a metal sheet 4100 is positioned on the back of the printed circuit board 4110. The sheet 4100 acts as a solid metal plane to shield electromagnetic fields from the coil 4120. Accordingly, other low magnitude signals tuned to the same operating frequency as the receiving circuit can be blocked, thus preventing accidental tripping of the therapeutic device into the ON or OFF state.

In some implementations, the preserver circuit also can include a modulation circuit that modulates the signal prior to amplification according to a known modulation process. For example, the signal can be modulated using on-off keying (OOK) modulation in which digital data is represented as the presence or absence of a carrier wave. This can be used to transmit a desired digital pattern or code to the receiving circuit, which can include a demodulator and memory. Upon receiving the signal, the demodulator can identify the pattern and send it to the comparator to be compared against a pattern stored in memory. If the pattern matches, the comparator can issue an activation signal to a switch (e.g., switch 3950 in FIG. 39) to activate or deactivate the therapeutic electromagnetic device.

In some implementations, the insole electromagnetic therapy device can include an internal battery that is rechargeable. FIG. 42 is a schematic of an example of base charging station 4200 for an insole electromagnetic therapy device. The device 4200 can include a rechargeable battery 4210, a base charge coil 4220 and a charging cable 4230 that couples to the battery 4210 and is operable to be inserted into a wall outlet for recharging the device 4200. In some implementations, the device 4200 also may include an LED 4240 to indicate when the rechargeable battery 4210 is fully recharged. The base charging station 4200 recharges an insole electromagnetic therapy device through electromagnetic induction. That is, when activated, the station 4200 transfers electromagnetic energy from the coil 4220 to an insole electromagnetic therapy device on or near the recharging station 4200. The charging station 4200 does not need to be plugged in when transferring energy to the insole therapy device. The electromagnetic field emitted by the recharging station 4200 can be received by the antenna of the electromagnetic therapy device. That is, the antenna of the electromagnetic therapy device can serve both as a receiver coil for the electromagnetic field from the recharging station and as an emitter to emit a therapeutic electromagnetic field to a user. The power from the received electromagnetic field then is transferred to the rechargeable battery of the insole electromagnetic device. Alternatively, the insole electromagnetic therapy device can include an additional coil for receiving the electromagnetic field, whereas the antenna would remain dedicated to emitting a therapeutic electromagnetic field to a user.

In some implementations, the charging station 4200 does not include the battery 4210, in which case the cable 4230 couples power to the coil 4220 when the cable 4230 is plugged into an AC outlet. The outer case of the charging station 4200 can be formed of a non-conductive, non-ferrous material so as not to impede the electromagnetic field being transferred to the insole therapy device. For example, the outer casing of the station 4200 can be formed from materials such as plastic or polycarbonate. Instead of transferring power to the electromagnetic therapy device using electromagnetic fields, power also can be transferred through direct contact of the electromagnetic therapy device to contact electrodes on the base station. In such implementations, the base station may or may not include the coil 4220. In either case, the electromagnetic therapy device can be recharged by connecting exposed positive and negative electrodes on the therapy device to a positive and negative electrode on the recharging station 4200. The positive and negative electrodes of the recharging station can be coupled to the internal battery 4210 of the station 4200 or to the cable 4230. The positive and negative electrodes of the therapy device can be connected to an internal battery of the therapy device. The electrodes of the electromagnetic therapy device can be accessed by removing the device from the insole gel coating.

A number of implementations have been described. Nevertheless, it will be understood that various modifications may be made without departing from the spirit and scope of the invention.

What is claimed is:

1. A device, comprising:
a therapeutic electromagnetic circuit configured to emit an electromagnetic field upon activation, wherein the therapeutic electromagnetic device comprises:
a circuit board, having an electromagnetic field generator thereon;
an antenna, coupled to the circuit board and arranged to radiate the electromagnetic field generated by the electromagnetic field generator;
a power source, coupled to the electromagnetic field generator via an activator; and
the activator, when turned on, initiating the electromagnetic field generator; and
a resilient insole coating surrounding the therapeutic electromagnetic circuit.

2. The device of claim 1, wherein the antenna is a single loop wire.

3. The device of claim 1, wherein the resilient shoe insole coating is composed of a gel material or rubber.

4. The device of claim 1, wherein the therapeutic electromagnetic circuit comprises an indicator operable to indicate an activation status of the device.

5. The device of claim 1, wherein the circuit board is integrated into an ASIC chip.

6. The device of claim 1, wherein the antenna has an asymmetrical shape.

7. The device of claim 1, wherein the antenna has a symmetrical shape.

8. The device of claim 2, wherein the single loop wire has a shape selected from a group consisted of a circle, an ellipse, and a rectangle.

9. The device of claim 2, wherein the single loop wire has a diameter thickness of about 20 gauge, is circle-shaped, and has a length ranging from about 3.14 mm to about 47.12 mm.

10. The device of claim 1, wherein the antenna is set on either side of the circuit board.

11. The device of claim 1, further comprising a hardened moisture resistant enclosure that encloses the circuit board, the power source, and the activator.

12. The device of claim 1 wherein the activator is a slide switch assembly comprising:
an injection molded switch channel;
an injection molded switch cover; and
a slide switch on the circuit board,
wherein the device further comprises means for protecting the slide switch assembly from accidental activation.

13. The device of claim 1 further comprising:
a hard potted enclosure layer;
wherein the circuit board includes circuit elements, metal dome switches, and a thin film layer substrate.

14. The device of claim 13 further comprising:
a light emitting diode indicator,
wherein a portion of the hard potted enclosure layer that corresponds to the light emitting diode indicator is transparent so that light from the light emitting diode indicator may permeate said hard potted enclosure and be seen by a user.

15. The device of claim 13 wherein the thin film layer substrate has a switch cut for a metalized dome switch; a metalized dome cavity for an on/off switch and two pull tab slits arranged on opposite sides of the metalized dome cavity along a common axis, wherein the two pull tab slits are configured for inserting a pull tab 1713.

16. The device of claim 1 further comprising:
a top plastic piece; and
a bottom plastic piece,
wherein the top plastic piece and the bottom plastic piece are configured to be engaged together by press-fitting pins on one of either the top plastic piece or the bottom plastic piece into recessed sockets on the other of either the top plastic piece or the bottom plastic piece.

17. The device of claim 1 further comprising a light emitting diode (LED) on the circuit board.

18. The device of claim 1 wherein the resilient insole coating is separable to allow access to the electromagnetic therapy device.

19. A system comprising:
a device according to claim 1; and
a recharging station to recharge the device.

20. The system of claim 19 wherein the recharging station comprises:
a battery; and
a first recharging coil coupled to the battery, wherein, during operation of the recharging system, the first recharging coil is operable to emit an electromagnetic field to recharge the device.

21. The system of claim 19 wherein the device comprises a second recharging coil to receive the electromagnetic field emitted by the first recharging coil.
